SEQUENCE LISTING (5 sequences)

SEQ ID NO: 1 - protein, 6 aa, Artificial Sequence
  Description of Artificial Sequence Synthetic peptide
PRRRRR

SEQ ID NO: 2 - protein, 10 aa, Artificial Sequence
  Description of Artificial Sequence Synthetic peptide
PRRRRSSRRP

SEQ ID NO: 3 - protein, 13 aa, Artificial Sequence
  Description of Artificial Sequence Synthetic peptide
ASRRRRRGGRRRR

SEQ ID NO: 4 - protein, 14 aa, Artificial Sequence
  Description of Artificial Sequence Synthetic peptide
VSRRRRRRGGRRRR

SEQ ID NO: 5 - protein, 11 aa, Human immunodeficiency virus
YGRKKRRQRRR

(19) United States
(12) Patent Application Publication
(10) Pub. No.: US 2010/0284982 A1
(43) Pub. Date: Nov. 11, 2010

(54) Erythrocyte-encapsulated L-asparaginase for enhanced acute lymphoblastic leukemia therapy

(76) Inventors: Victor C. Yang, Ann Arbor, MI (US); Young M. Kwon, Ann Arbor, MI (US); Hee S. Chung, Ann Arbor, MI (US); Arthur J. Yang, York, PA (US)

Correspondence Address: JONES DAY, 222 EAST 41ST ST, NEW YORK, NY, 10017, US

(21) Appl. No.: 12/318,112

(22) Filed: Dec. 22, 2008

Related U.S. Application Data
(60) Provisional application No. 61/008,288, filed on Dec. 20, 2007.
(60) Provisional application No. 61/064,650, filed on Mar. 18, 2008.

(51) Int. Cl. A61K 35/18; A61P 35/00; A61P 35/02; C12N 9/82; C12N 9/96

(52) U.S. Cl. 424/93.73; 435/229; 435/188

(57) ABSTRACT

Compositions for transporting L-asparaginase across the cellular membrane of erythrocytes, comprising a low molecular weight protamine peptide. Process of preparation of compositions comprising conjugates of L-asparaginase and a low molecular weight protamine peptide. Method of treatment comprising administration of adapted L-asparaginase is also described.

### CROSS REFERENCE TO RELATED APPLICATIONS

This application claims the benefit of priority from U.S. Provisional Application No. 61/008,288, filed Dec. 20, 2007, and from U.S. Provisional Application No. 61/064,650, filed Mar. 18, 2008. These applications, in their entirety, are both incorporated herein by reference. This application also incorporates by reference U.S. patent application Ser. No. 10/548,438, filed Sep. 14, 2006, in its entirety.

The work of this application was supported by grant number 1R43CA135969-01 from the United States National Institutes of Health. Thus, the U.S. government may have certain rights in the invention.

### SUMMARY OF THE INVENTION

In one aspect of the invention, the low molecular weight protamine peptide comprises at least 5 amino acids of the following amino acid sequence: VSRRRRRRGGRRRR (SEQ ID NO: 4). For example, at least 6 amino acids, at least 7 amino acids, at least 8 amino acids, at least 9 amino acids, at least 10 amino acids, at least 11 amino acids, at least 12 amino acids, at least 13 amino acids, or at least 14 amino acids.

In one aspect of the invention, the purity of the low molecular weight protamine peptide may be 50-100%. For example, the purity may be at least 50%, at least 60%, at least 70%, at least 80%, at least 85%, at least 90%, at least 95%, at least 98%, at least 99%, or at least 99.99%.

In one aspect of the invention, the conjugate comprises L-asparaginase and the low molecular weight protamine peptide. The homology of the low molecular weight protamine peptide, may have for example, at least 85%, at least 90%, at least 95%, at least 98%, or be or be identical to the amino acid sequence VSRRRRRRGGRRRR (SEQ ID NO: 4).

In one aspect of the invention, the conjugation of the L-asparaginase to the low molecular weight protamine peptide, comprises, for example, a covalent bond. The covalent bond, for example, comprises a disulfide bond, an amide bond, or an ether bond. The conjugation may further comprise a linker moiety, for example, a polymeric linker.

In one aspect of the invention, the erythrocyte comprises L-asparaginase and the low molecular weight protamine peptide. For example, the L-asparaginase may be conjugated to one or more of the low molecular weight protamine peptides.

In one aspect of the invention, the quantifiable amount of erythrocytes comprising L-asparaginase after treatment of an erythrocyte population with an adapted L-asparaginase may be 10-100% of the treated erythrocytes. For example, the quantifiable amount of erythrocytes treated may be at least 10%, at least 15%, at least 20%, at least 25%, at least 30%, at least 40%, at least 50%, at least 60%, at least 70%, at least 75%, at least 80%, at least 85%, at least 90%, at least 95%, at least 97%, at least 98%, at least 99%, or the entire population of treated erythrocytes.

In one aspect of the invention, the quantifiable amount of erythrocytes comprising L-asparaginase after treatment of an erythrocyte population with a conjugate that is or was conjugated to the low molecular weight protamine peptide may be 10-100% of the treated erythrocytes. For example, the quantifiable amount of erythrocytes treated may be at least 10%, at least 15%, at least 20%, at least 25%, at least 30%, at least 40%, at least 50%, at least 60%, at least 70%, at least 75%, at least 80%, at least 85%, at least 90%, at least 95%, at least 97%, at least 98%, at least 99%, or the entire population of treated erythrocytes.

In one aspect of the invention, the individual treated erythrocyte and/or each erythrocyte within an erythrocyte population is/are treated with L-asparaginase that is or was conjugated to the low molecular weight protamine peptide. For example, at least 5 amino acids, at least 6 amino acids, at least 7 amino acids, at least 8 amino acids, at least 9 amino acids, at least 10 amino acids, at least 11 amino acids, at least 12 amino acids, at least 13 amino acids. or at least 14 amino acids.

In one aspect of the invention, the individual treated erythrocyte and/or each erythrocyte within an erythrocyte population is/are treated with an adapted L-asparaginase. For example, at least 5 amino acids, at least 6 amino acids, at least 7 amino acids, at least 8 amino acids, at least 9 amino acids, at least 10 amino acids, at least 11 amino acids, at least 12 amino acids, at least 13 amino acids. or at least 14 amino acids.

In one aspect of the invention, the individual treated erythrocyte and/or each erythrocyte within an erythrocyte population that is/are treated with a conjugate comprises conjugates wherein the number of low molecular weight protamine peptides that is or was conjugated to the L-asparaginase, comprises at least one per L-asparaginase. For example, at least two, at least three, at least four, at least five, at least six, at least seven, or at least eight low molecular weight protamine peptides that is/are or was conjugated to the L-asparaginase. Preferably between, for example, 1-8, 2-7, 3-6, or 4-5 low molecular weight protamine peptides per L-asparaginase.

In one aspect of the invention, the individual treated erythrocyte and/or erythrocyte population may retain between 50-100% of at least one of the attributes and/or functionality, as compared to a reference control or to a normal, untreated erythrocyte and/or erythrocyte population. For example, at least 50%, at least 55%, at least 60%, at least 65%, at least 70%, at least 75%, at least 80%, at least 85%, at least 95%, at least 95%, at least 96%, at least 97%, at least 98%, at least 99% or 100% retention of at least one of the attributes and/or functionality as compared to a normal, untreated erythrocyte and/or erythrocyte population.

In one aspect of the invention, the individual treated erythrocyte and/or erythrocyte population may be within 0.5-20% of at least one of the attributes and/or functionality, as compared to a reference control or to a normal, untreated erythrocyte and/or erythrocyte population. For example, within 20%, within 15%, within 10%, within 9%, within 8%, within 7%, within 6%, within 5%, within 4.5%, within 4%, within 3.5%, within 3.4%, within 3.3%, within 3.2%, within 3.1%, within 3%, within 2.9%, within 2.8%, within 2.7%, within 2.6%, within 2.5%, within 2%, within 1.5%, within 1%, within 0.8%, within 0.7%, within 0.6%, or within 0.5% retention of at least one of the attributes and/or functionality as compared to a reference control or a normal, untreated erythrocyte and/or erythrocyte population.

In one aspect of the invention, the representative attributes and/or functionality of normal, untreated erythrocyte and/or erythrocyte population that may be retained by the erythrocyte and/or erythrocyte population, comprises, but is not limited to the following:

i) structural integrity of the erythrocyte(s);

ii) biological and/or morphological integrity of the erythrocyte(s), comprising hematological values, comprising:

a) mean cell volume;

b) mean cell hemoglobin;

c) mean cell hemoglobin content;

iii) oxygen transport activity of the erythrocyte(s);

iv) oxygen dissociation of the erythrocyte(s);

v) osmotic fragility of the erythrocyte(s);

vi) biochemical attributes of the erythrocyte(s);

vii) chemical attributes of the erythrocyte(s);

viii) energy (ATP)-involved metabolic activity of the erythrocyte(s); and/or

viiii) scavenging of oxidative stress activity of the erythrocyte(s).

In one aspect of the invention, the composition, comprises L-asparaginase having the biological property to translocate across a biological membrane of a cell. The biological membrane, for example, may be the cellular membrane or the membrane of an organelle within the cell. The cell, for example, may be an erythrocyte. The composition may comprise L-asparaginase and the low molecular weight protamine peptide. The composition may comprise L-asparaginase that is or was conjugated to the low molecular weight protamine peptide. The composition may comprise an adapted L-asparaginase.

In one aspect of the invention, an adapted L-asparaginase may have the biological property to translocate across a biological membrane of a cell. The biological membrane, for example, may be the cellular membrane or the membrane of an organelle within the cell. The cell, for example, may be an erythrocyte. The adapted L-asparaginase may comprise L-asparaginase and the low molecular weight protamine peptide. The adapted L-asparaginase may comprise L-asparaginase that is or was conjugated to the low molecular weight protamine peptide.

In one aspect of the invention, the individual treated erythrocyte and/or erythrocyte population, once comprising the L-asparaginase, may substantially maintain at least one of the following properties: structural integrity; biological integrity; morphological integrity; and functionality. The individual treated erythrocyte and/or erythrocyte population, wherein the erythrocyte may substantially maintain at least its structural integrity and at least one of biological integrity, morphological integrity, and functionality. The individual treated erythrocyte and/or erythrocyte population, wherein the treated erythrocyte may substantially maintain at least its structural integrity and functionality, and at least one of biological integrity and morphological integrity.

In one aspect of the invention, the composition, comprising L-asparaginase having the biological property to translocate across a biological membrane of a cell, once comprising the L-asparaginase, may substantially maintain at least one of the following properties: structural integrity; biological integrity; morphological integrity; and functionality. The composition, comprising L-asparaginase wherein the erythrocyte may substantially maintain at least its structural integrity and at least one of biological integrity, morphological integrity, and functionality. The composition, comprising L-asparaginase wherein the treated erythrocyte may substantially maintain at least its structural integrity and functionality, and at least one of biological integrity and morphological integrity.

In one aspect of the invention, the L-asparaginase adapted to having the biological property to translocate across a biological membrane of a cell, once comprising the L-asparaginase, may substantially maintain at least one of the following properties: structural integrity; biological integrity; morphological integrity; and functionality. The adapted L-asparaginase, wherein the erythrocyte may substantially maintain at least its structural integrity and at least one of biological integrity, morphological integrity, and functionality. The adapted L-asparaginase, wherein the treated erythrocyte may substantially maintain at least its structural integrity and functionality, and at least one of biological integrity and morphological integrity.

In one aspect of the invention, the method of preparing an individual erythrocyte comprising L-asparaginase, may comprise: conjugating L-asparaginase and the low molecular weight protamine peptide.

In one aspect of the invention, the method of preparing an erythrocyte population, comprising a quantifiable amount of erythrocytes having L-asparaginase, may comprise: conjugating L-asparaginase and the low molecular weight protamine peptide.

In one aspect of the invention, the process of treating a patient suffering from cancer, may comprise: administering a therapeutic amount of erthrocytes comprising L-asparaginase to said patient. The process of administering L-asparaginase, for example, may comprise administering L-asparaginase that is or was conjugated to the low molecular weight protamine peptide. The process of administering L-asparaginase may administer the L-asparaginase by lysing of the L-asparaginase from the conjugate. The process may comprise: extracting a population of erythrocytes from said patient, treating the extracted population of erythrocytes with L-asparaginase, and administering L-asparaginase that is or was conjugated to the low molecular weight protamine peptide. The process may comprise treatment of the extracted erythrocytes with L-asparaginase that is or was conjugated to the low molecular weight protamine peptide. The patient suffering from cancer may be suffering from other proliferative diseases, for example, leukemia.

In one aspect of the invention, the process of treating a patient suffering from cancer, may comprise: administering a therapeutic amount of erthrocytes comprising L-asparaginase to said patient. The processs of treatment, for example, may comprise: i) forming a conjugate, comprising: a) one or more low molecular weight protamine peptides, wherein at least one of the peptides having at least 80% homology to the amino acid sequence VSRRRRRRGGRRRR (SEQ ID NO: 4); and b) L-asparaginase; ii) treating an erythrocyte population with said conjugate; and iii) administering the conjugate treated erythrocyte population to said patient.

### Additional Exemplary Embodiments of the Present Invention

A. An erythrocyte, comprising:

i) a low molecular weight protamine peptide having at least 80% homology to the amino acid sequence VSRRRRRRGGRRRR (SEQ ID NO: 4); and

ii) L-asparaginase.

B. The embodiment of A, wherein the L-asparaginase is conjugated to the low molecular weight protamine peptide.

C. The embodiment of B, wherein the homology is at least 90%.

D. An erythrocyte population, comprising a quantifiable amount of erythrocytes, comprising:

i) a low molecular weight protamine peptide having at least 80% homology to the amino acid sequence VSRRRRRRGGRRRR (SEQ ID NO: 4); and

ii) L-asparaginase.

E. The embodiment of D, wherein the L-asparaginase is conjugated to the peptide.

F. The embodiment of E, wherein the conjugate comprises at least one said low molecular weight protamine peptide per L-asparaginase.

G. The embodiment of F, wherein the conjugate comprises 3-6 said peptide per L-asparaginase.

H. The embodiment of G, wherein the peptide comprises at least 14 amino acids, wherein the amino acid sequence of said peptide, comprises:

| | |
|---|---|
| VSRRRRRRGGRRRR. | (SEQ ID NO: 4) |

I. The embodiment of H, wherein said population retains at least 50% of least one of the attributes and/or functionality, as compared to a normal, untreated erythrocyte population.

J. The embodiment of I, wherein said attributes and/or functionality, comprises:

i) structural integrity of the erythrocytes;

ii) biological and/or morphological integrity of the erythrocytes, comprising hematological values, comprising:

a) mean cell volume;

b) mean cell hemoglobin;

c) mean cell hemoglobin content;

iii) oxygen transport activity;

iv) oxygen dissociation;

v) osmotic fragility;

vi) biochemical attributes;

vii) chemical attributes;

viii) energy (ATP)-involved metabolic activity; and/or

viiii) scavenging of oxidative stress activity.

K. The embodiment of J, wherein said erythrocyte population comprises a single erythrocyte.

L. A composition, comprising an L-asparaginase having the biological property to translocate across a biological membrane of an erythrocyte.

M. The embodiment of L, wherein said composition comprises the L-asparaginase that is or was a conjugate to at least one low molecular weight protamine peptide having at least 80% homology to the amino acid sequence VSRRRRRRGGRRRR (SEQ ID NO: 4).

N. The embodiment of M, wherein said erythrocyte, once comprising the L-asparaginase, substantially maintains at least one of the following properties:

i) structural integrity;

ii) biological integrity;

iii) morphological integrity; and

iv) functionality.

O. The embodiment of N, wherein said erythrocyte substantially maintains at least its structural integrity and at least one of biological integrity, morphological integrity, and functionality.

P. A process of treating a patient suffering from cancer, comprising:

administering a therapeutic amount of erthrocytes comprising L-asparaginase to said patient.

Q. The embodiment of P, wherein the L-asparaginase administered is or was conjugated to a low molecular weight protamine peptide having at least 80% homology to the amino acid sequence VSRRRRRRGGRRRR (SEQ ID NO: 4).

R. The embodiment of Q, wherein the L-asparaginase is lysated from said conjugate.

S. The embodiment of R, wherein prior to the therapeutic administering of said erythrocytes, said process further comprises:

i) extracting a population of erythrocytes from said patient; and

ii) treating the extracted population of erythrocytes with L-asparaginase;

T. The embodiment of S, wherein the L-asparaginase used to treat the extracted erythrocytes is or was conjugated to a low molecular weight protamine peptide having at least 80% homology to SEQ ID NO: 4.

U. The embodiment of T, wherein the L-asparaginase used to treat the extracted erythrocytes is conjugated to a low molecular weight protamine peptide having at least 80% homology to SEQ ID NO: 4.

V. The embodiment of U, wherein the patient is suffering from leukemia.

W. The embodiment of V, wherein said process comprises

i) forming a conjugate, comprising:

a) one or more low molecular weight protamine peptides, wherein at least one of the peptides having at least 80% homology to the amino acid sequence VSRRRRRRGGRRRR (SEQ ID NO: 4); and

b) L-asparaginase;

ii) treating an erythrocyte population with said conjugate; and

iii) administering the conjugate treated erythrocyte population to said patient.

X. The process of treating a patient suffering from leukemia, comprising the L-asparaginase administered is or was conjugated to a low molecular weight protamine peptide having at least 80% homology to the amino acid sequence VSRRRRRRGGRRRR (SEQ ID NO: 4).

Y. The embodiment of X, wherein:

administering L-asparaginase is conjugated to a low molecular weight protamine peptide having at least 80% homology to the amino acid sequence VSRRRRRRGGRRRR (SEQ ID NO: 4) to said patient.

Z. The embodiment of Y, wherein:

i) extracting a population of erythrocytes from said patient;

ii) treating the extracted population of erythrocytes with L-asparaginase;

iii) administering said treated population of erythrocytes to said patient.

AA. The embodiment of Z, wherein the L-asparaginase is lysated from a conjugate comprising L-asparaginase and a low molecular weight protamine peptide having at least 80% homology to the amino acid sequence VSRRRRRRGGRRRR (SEQ ID NO: 4).

BB. The embodiment of AA, wherein said population of erythrocytes were drawn from said patient.

CC. The embodiment of BB, wherein said process comprises:

i) treating a population of erythrocytes with L-asparaginase conjugated to a low molecular weight protamine peptide having at least 80% homology to SEQ ID NO: 4; and

ii) administering treated population of erythrocytes to same said patient.

DD. The embodiment of CC, wherein said population of erythrocytes were drawn from said patient.

EE. A method of preparing an erythrocyte having L-asparaginase, comprising:

conjugating L-asparaginase and a low molecular weight protamine peptide having at least 80% homology to the amino acid sequence VSRRRRRRGGRRRR (SEQ ID NO: 4).

FF. A method of preparing an erythrocyte population, comprising a quantifiable amount of erythrocytes having L-asparaginase, comprising:

conjugating L-asparaginase and a low molecular weight protamine peptide having at least 80% homology to the amino acid sequence VSRRRRRRGGRRRR (SEQ ID NO: 4).

GG. An L-asparaginase adapted to having the biological property to translocate across a biological membrane of an erythrocyte.

HH. The embodiment of GG, wherein said adapted L-asparaginase is or was a conjugated to at least one low molecular weight protamine peptide having at least 80% homology to the amino acid sequence VSRRRRRRGGRRRR (SEQ ID NO: 4).

II. The embodiment of HH, wherein said erythrocyte, once comprising the L-asparaginase, substantially maintains at least one of the following properties:

i) structural integrity;

ii) biological integrity;

iii) morphological integrity; and

iv) functionality.

JJ. The embodiment of II, wherein said erythrocyte substantially maintains at least its structural integrity and at least one of biological integrity, morphological integrity, and functionality.

### BRIEF DESCRIPTION OF THE DRAWINGS

FIG. 1(a) is a SEM of native RBCs with normal discocyte form processed by hypotonic loading;

FIG. 1(b) is a SEM of processed RBCs with abnormal stomatocyte form processed by hypotonic loading;

FIG. 1(c) is a SEM of native RBCs with normal discocyte form processed by electroporation;

FIG. 1(d) is a SEM of processed RBCs with abnormal stomatocyte form processed by electroporation;

FIG. 2 is a schematic illustration of the proposed RBC encapsulation technology;

FIG. 3 shows the conversion of asparagine (ASN) to aspartic acid (ASP) by RBC-encapsulated asparaginase (ASNase);

FIG. 4 shows the cellular translocation of LMWP-FITC conjugates into cultured HeLa cells after 30 minutes of in vitro incubation with a differential interference contrast image (left) and a fluorescence microscopy (right);

FIG. 5 shows cellular localization of rhodamine-labeled phalloidin-LMWP conjugates in MG63 cells after 30 min incubation at 37° C.;

FIG. 6 is a flow cytometric analysis of AlexFluor-488 labeled phalloidin, LMWP-phalloidin, and TAT-phalloidin in MG63 osteoblast cells after incubation at 37° C. for 30 min in a medium containing 10% serum;

FIG. 7 are confocol microscopic images of untreated RBC (Control RBC; first row); RBC incubated with Alex a Fluor 488-labeled ovalbumin (OVA-488, second row); and LMWP-ovalbumin (LMWP-OVA-488; third row), wherein the first column is fluorescence mode, the second column is DIC mode and third column is superimposition;

FIG. 8 is a MALDI-TOF mass spectra of the chemically synthesized LMWP-ASNase conjugates;

FIG. 9 are scanning electron microscopic images of (A) Untreated normal RBCs (i.e. Control); and (B) LMWP-ASNase-.encapsulated RBCs;

FIG. 10 shows osmotic fragility curves for normal (red line) and ASNase-loaded (green line) RBCs. Osmotic fragility was performed according to procedures described in Section D.1.3.2 below;

FIG. 11 shows pharmacokinetic profiles of ASNase activity in blood in DBA2 mice. The red data points represent RBCs loaded with ASNase via the hypotonic method, whereas the green data points represent RBCs loaded with ASNase via the proposed LMWP-mediated encapsulation method. ASNase-loaded RBCs (8 IU/mouse) were administered via tail vein injection. ASNase activity in whole blood was monitored by nesslerization. Each experimental group consisted of 4 animals;

FIG. 12 is a Kaplan-Meier survival curve for DBA/2 mice bearing L5178Y lymphoma cells. Saline solution (Control) or ASNase-loaded RBCs (8 IU/mouse; Experimental) were administered via tail vein injection on Day 5, at which symptom was apparent. Three animals were involved in each test group;

FIG. 13 shows LMWP-ASNase stability at 4 and 37° C.;

FIG. 14 shows torage stability of LMWP-ASNase at 4, −20, and −80° C.;

FIG. 15 is the effect of temperature on LMWP-ASNase loading into RBCs;

FIG. 16 shows loading kinetics of LMWP-ASNase into RBCs;

FIG. 17 shows SEM images of control (sham loaded) and LMWP-ASNase loaded mouse RBCs;

FIG. 18 shows osmotic fragility curves of control (sham loaded) and LMWP-ASNase loaded sheep RBCs; and

FIG. 19 is an enzyme concentration curve for varying incubating solutions.

### DETAILED DESCRIPTION OF THE INVENTION

Acute lymphoblastic leukemia (ALL) is cancer of the white blood cells, the cells that normally fight infections. Approximately 4,000 new cases of ALL are diagnosed annually in the United States alone, and two thirds of which are in children and adolescents, making ALL the most common cancer in these age groups. One of the primary drugs used in treatment of ALL is L-asparaginase (ASNase), which induces systemic depletion of asparagine (ASN)—an amino acid that is essential for the survival of ALL cells. Despite its wide use, the clinical application of ASNase faces two obstacles. First, ASNase is a non-human, immunogenic protein and its clinical use is therefore associated with significant anaphylactic responses. Additionally, like most protein drugs, ASNase is susceptible to degradation by serum proteases and clearance by the reticuloendothelial system (RES). Consequently, the plasma half-life of ASNase is relatively short, within the range of 8-30 hours. This short half-life demands frequent injection of large doses of the drug, further increasing the risk of inducing severe allergic responses. To overcome such problems, extensive efforts have been focused on the protection of ASNase with either a synthetic (e.g. polymers) or natural (e.g. cells) carrier. Among all the drug carriers, red blood cells (RBCs) appears to be the most appealing, because they are completely biocompatible and biodegradable, and also possess a life-span (120 days) that is unmatchable by other existing carriers. A variety of techniques have successfully entrapped protein drugs into RBCs. However, these methods all require the disruption of the RBC membranes with a certain degree of chemical (e.g. drug-induced endocytosis), mechanical (hypotonic osmosis/dialysis), or electrical (electroporation) force to create large pores for proteins drugs to diffuse in. Unfortunately, these forces cause membrane deterioration and, as a consequence, result in a loss of structural integrity and cellular components of the erythrocytes, rendering them prone to destruction or recognition by the host immune system. It should be specifically pointed out that in order to inherit the benefits of RBC as a natural and long-circulating drug carrier, it is absolutely essential to retain complete structural and functional integrity of the erythrocytes; all of the existing RBC encapsulation methods fail to meet this critical point.

Recently, a family of small, extraordinarily potent cell-penetrating PTD (protein transduction domain) peptides has been discovered. Both in vitro and animal studies revealed that PTD was able to ferry covalently attached macromolecular species, including proteins and drug carriers, across cell membranes of all organ types including the brain. Remarkably, the PTD-mediated cell internalization process does not appear to induce any perturbation or alteration of the cell membrane. Importantly, one of the PTD peptides developed by ISTN scientists and Prof. Yang's (Co-PI of this project) research group at the University of Michigan, Low Molecular Weight Protamine (LMWP), was demonstrated in extensive animal studies to be neither toxic nor immunogenic. These desirable features originated the conceptual framework of the proposed non-invasive RBC-encapsulation technology for ASNase. Briefly, the approach calls for covalent conjugation of ASNase with LMWP via disulfide linkages. Because of the universal and potent membrane-penetrating activity of LMWP, even without the aid of surface-disrupting forces, the LMWP-ASNase conjugates should be able to internalize into erythrocytes without altering the RBC's structural and/or functional attributes. Within the cells, LMWP would, by design, dissociate from its protein counterpart via degradation of the disulfide linkage, due to the existence of a high level of cytoplasmic glutathione and reductase activities. This bond dissociation would enable the cell-impermeable ASNase to remain permanently entrapped in the erythrocytes, ensuring full protection of ASNase from detection by the host immune system and clearance by RES and other endogenous factors. Since it has already been demonstrated clinically that the substrate asparagine (ASN) is capable of permeating human RBCs freely, the ASNase-encapsulated erythrocytes would function as a live bioreactor, depleting ASN from the circulation and depriving leukemic cells of their essential nutrients and, consequently, leading to cell death. If both the physical and biological attributes of the RBC can be fully retained after the encapsulation process, then the RBC-entrapped ASNase would presumably retain the same circulating life-span of native erythrocytes (i.e. 120 days), yielding longer-lasting therapeutic effect than that of ASNase delivered by other mechanisms. This would provide a dramatically reduced dosing frequency (by more than 100 fold) required for achieving an effective anti-ALL therapy, significantly alleviating the toxic side effects associated with current ASNase therapies.

Extremely promising results have been obtained during the course of preliminary investigation. RBCs processed by this novel encapsulation technology exhibited an intact structure and functionality that were indistinguishable from those of the normal, untreated RBCs. In vivo findings were also in full agreement with these in vitro results, as the RBC-entrapped ASNase not only inherited an exceedingly prolonged plasma half-life in healthy mice, but also displayed potent and long-lasting therapeutic effects in mice harboring the leukemic cells. In this Phase I research, we plan to build on these exciting preliminary findings and carry out further proof-of-concept animal studies to completely validate the plausibility of this RBC-encapsulation technology. Four integrated specific aims are being planned: [1] further evaluation of the pharmacokinetic profiles of both normal RBCs and ASNase-loaded RBCs; [2] assessment of the therapeutic benefits of the RBC-loaded ASNase; [3] testing of the immunogenicity of the RBC-ASNase; and [4] examination of the toxicity of the RBC-ASNase system. Once the feasibility of the system is confirmed, a greatly extended Phase II application will be submitted. During Phase II, we plan to: [i] produce mass quantities of the LMWP-ASNase conjugates via a recombinant method; [ii] establish GMP procedures for the preparation of RBC-encapsulated ASNase; and [iii] carry out extensive animal studies related to the efficacy, safety, pharmacokinetics, pharmacodynamics, etc., to further develop this RBC-ASNase technology into a real clinical remedy.

Acute lymphoblastic leukemia (ALL) is a type of cancer in which the bone marrow makes too many immature white blood cells called lymphocytes that are unable to help the body fight infections. As the number of lymphocytes increases in the blood and bone marrow, there is also less room for healthy white blood cells, red blood cells, and platelets. As a consequence, ALL patients often suffer infections, anemia, and easy bleeding. Almost 4,000 cases of ALL are diagnosed annually in the United States alone, approximately two thirds of which are in adolescent children, making ALL the most common cancer in this age group. Indeed, ALL represents 23% of the cancers diagnosed among children younger than 15 years of age, occurring at an annual rate of 30 to 40 per million. While a cure rate of ˜80% was estimated for childhood ALL, the experience with adult ALL was far less rewarding, as the reported cure rate seldom exceeded 40%.

One of the primary drugs used in treatment of ALL is L-asparaginase (ASNase), which has been in clinical use since 1967. ASNase is an enzyme which hydrolyzes amino acid L-asparagine (ASN) into L-aspartic acid and ammonia. Most human tissues can self-synthesize L-asparagine from L-glutamine by the action of asparagine synthase (AS). Certain neoplastic tissues, including ALL cells, however, express a significantly lower level of AS and thus have to rely solely on an extracellular source of L-asparagine to maintain protein synthesis. Systemic depletion of ASN by ASNase would therefore impair protein biosynthesis and, subsequent, arrest the cell cycle in these cells, leading to their deaths through cellular dysfunction.

ASNase formulations currently in clinical use are originated from two bacterial sources, Escherichia coli and Erwinia chrysanthemi. The enzyme is a tetramer, with each monomer containing an active site, and has an overall molecular weight of 133-140 kDa. The specific activity of purified ASNase ranges between 300-400 □mole of substrate/min/mg of protein. The isoelectric point lies between pH 4.5-5.5 for the E. coli enzyme and 8.6 for the Erwinia enzyme. The Kₘ is approximately 1×10⁻⁵ M. ASNase is not adsorbed from the GI track, and thus in clinical practice, it is normally administered via the intravenous or intramuscular route.

Like most protein drugs, clinical application of ASNase faces two major obstacles. First, ASNase is a non-human protein, and its clinical use is therefore associated with a high incidence of hypersensitive reactions. Specifically, with its bacterial origin, ASNase is capable of triggering significant immunological consequences including activation of B lymphocytes and production of antibodies, causing severe anaphylactic reactions and, in certain cases, even fatal consequences. Most allergic reactions occur within one to several hours after drug administration and include signs and symptoms typical of anaphylaxis.

Secondly, like most protein drugs, ASNase is susceptible to degradation by serum proteases and elimination by the reticuloendothelial system (RES). The plasma half-life of ASNase, which is not related to dose or organ (e.g. liver, kidney, etc.) function, is estimated to be in the range of 8-30 hours. This rapid body clearance demands frequent injection of large doses of ASNase, further elevating the chance of inducing severe immunological responses.

To overcome such problems of short circulating half-life and immunogenic reactions of ASNase, various approaches have been attempted. The most successful or commonly employed methods to-date include incorporation of hydrophilic polyethylene glycol (PEG) moieties to this protein drug (a process termed “pegylation”), or encapsulation of ASNase into soluble, synthetic (e.g. polymers) or natural (e.g. liposomes, cells), carriers. Attaching polyethylene glycol (PEG) chains increases the mass of the enzyme drug and shields it from proteolytic degradation, improving pharmacokinetics of the drug. Indeed, PEG-modified ASNase, with a trademark name of pegaspargase, has been successfully developed during 1970s, with first clinical trial being carried out in 1980s. Clinical results showed that the half-life of pegaspargase was extended from the original 26 hrs for the free enzyme to about 15 days. In addition, this new form of ASNase was better tolerated than the free form, especially when given intramuscularly. Hence, pegaspargase has been specifically indicated for treating ALL patients who are sensitive to native ASNase. According to a review of clinical data, in re-induction therapy for patients who were hypersensitive to E. coli-derived ASNase, pegaspargase was able to reduce the frequency of drug administration from 6-9 to 1-2 times per therapy. Nevertheless, pegaspargase has not yet been demonstrated to be superior to E. coli ASNase for the first remission of ALL.

Among all carriers employed for ASNase encapsulation, the use of erythrocytes (red blood cells; RBCs) as the drug carrier appears to be most appealing, simply because the erythrocyte would not only protect the loaded protein drug from proteolytic degradation but also prevent detection of the drug by the host immune system. Furthermore, erythrocytes are completely biodegradable without generation of toxic products, and they are also biocompatible, particularly when autologous erythrocytes are used. In addition, erythrocytes are the most abundant cells of the human body (5.4×10⁶ and 4.8×10⁶ RBCs/mL in men and women, respectively), therefore giving an affordable source of supply for use in drug encapsulation. Moreover, the biconcave disk shape of erythrocytes endows them with the highest surface to volume ratio (1.9×10⁴ cm/g) usable for drug encapsulation. Most critically, erythrocytes possess a lifespan in circulation of approximately 120 days, which is significantly longer than any of the presently existing drug carriers. A detailed examination of the benefits of utilizing erythrocytes as the drug carrier can be found in a review article authored by Hamidi and Tajerzadeh.

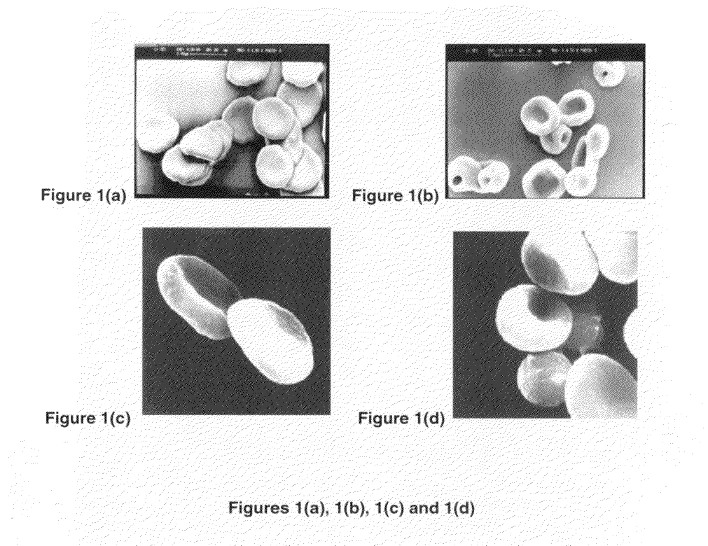

A variety of methods have already been developed to entrap protein drugs into RBCs. The most adapted techniques thus far include drug (e.g. primaquine, hydrocortisone, etc.)—induced endocytosis, electroporatio, and hypoosmotic-based preswelling, rupture/resealing, or dialysis. Using these methods to create sufficiently large pores or perturbations on the cell membrane, a number of the impermeable protein drugs including L-asparaginase, erythropoietin, acetaldyhyde dehydrogenase and alcohol dehydrogenase have been successfully loaded into RBCs. Despite reasonable success, all of these methods are still beset by a host of shortcomings. The most crucial drawbacks come from two aspects following RBC processing. First, these techniques all require the application of a chemical (drug-induced endocytosis), electrical (electroporation), or mechanical (osmotic dialysis) force to the RBC membrane to create sufficiently large pores for the protein drug to diffuse through. Such disruption of the cell membrane often leads to partial but irreversible deterioration of the structural integrity and morphology of the erythrocyte. As displayed in FIG. 1, a significant alteration of the erythrocyte morphology from the native discocyte form (i.e. normal erythrocyte with a small area of central pallor biconcave disc shape; right panels of both pictures) to stomatocyte (i.e. abnormal erythrocyte with oval or rectangular area of central pallor; left panels of both pictures) following treatment by electroporation (top column) or hypo-osmosis (bottom column) was clearly observed. Consequently, these processed RBCs will be recognized by the phagocytic system as foreign entities, rendering their rapid destruction and clearance by the host immune system.

The second issue is that erythrocytes processed by any of the existing protein-loading methods, regardless whether it is electroporation or hypotonic dialysis, would inevitably result in a loss of important cellular constituents, such as hemoglobin and cytoskeleton, from the cells. This is because all of these methods rely on a pore-opening and a resealing step, both of which involve a dialysis procedure. Thus far, the largest protein encapsulated in RBCs by using such methods was alcohol oxidase from Pichea pastoria, which had a molecular weight (675 kDa) that was 10-fold larger than that of hemoglobin (65 KDa); the major constituent of an erythrocyte. Since dialysis is an equilibrium process and with such large pores being created on the cell membrane, in theory and practice, it is inevitable that a certain portion of the cytosolic constituents including hemoglobin, glutathione, and cytoskeleton would be leaked out of the erythrocyte. Indeed, loss of hemoglobin was clearly observed in erythrocytes treated with the hypo-osmosis dialysis method, as evidenced by a decrease in MCH after resealing. It should be noted that aside from the principal activity of oxygen transport, RBCs also carry out other important biological functions, such as energy (ATP)-involved metabolic processes as well as scavenging of oxidative stress. Hence, a loss of hemoglobin would not only impair the oxygen transport function of RBCs, but also affect their ability to manage oxidative stress. Similarly, a loss of cytoskeleton from the erythrocyte would compromise it with a much weakened structural integrity, rendering it prone to destruction or recognition by the phagocytic system. It is important to point out that in order to inherit the benefits of RBC as a natural and long-lasting drug carrier, it is absolutely essential to retain both the structural and functional integrity of the cell. Yet, all of the existing RBC encapsulation methods fail to recognize this critical point. Therefore, the need of a method that would permit encapsulation of therapeutically active protein drugs into fully functional erythrocytes persists, and the quest continues.

Recently, the discovery of a family of small but extraordinarily potent cell-penetrating peptides (CCP; also widely termed as PTD (protein transduction domain) peptides) that includes TAT, ANTP, VP22, arginine-rich peptides, and the non-toxic, naturally occurring low molecular weight protamine (LMWP) peptide have drawn significant attention from the scientific community. Both in vitro and animal studies revealed that, by covalently linking PTD to almost any type of molecular species, including proteins (MW>150 kDa; more than 60 different proteins have already been tested) and nano-carriers (e.g. liposomes), PTD was able to ferry the attached species across cell membranes of all organ types including the brain. Most importantly, it was documented that PTD itself was relatively non-toxic and non-immunogenic, and PTD-mediated cell internalization did not induce perturbation or alteration of the cell membrane.

Since its establishment in 1997, ISTN has been actively involved in the synthesis and development of novel nano-structured biomaterials, by utilizing its leading and patented technologies to control morphology at supramolecular length scales. Most recently, ISTN has been working on the development of silica/chitosan-based nanocomposite for specific targeting of the stomach in treatment of peptic ulcers. Aside from this main area of biomedical application of silica-based, nanoporous composite materials, ISTN has also been involved in the development of low molecular weight protamine (LMWP) for potential clinical use as a highly effective, non-toxic antagonist to heparin and low molecular weight heparin (LMWH). Extensive progress has been made to demonstrate that LMWP was neither immunogenic (i.e. the ability to induce antibody production) nor antigenic (i.e. the ability to be recognized by the antibodies). In addition, unlike most commonly encountered highly cationic peptides, administration of LMWP into dogs did not elicit acute hypotensive responses or other toxicity such as complement activation.

Recently, in an important discovery reported by the FASEB Journal, ISTN scientists further demonstrated that since LMWP shared significant sequence similarity with TAT (see Table 1), the most established PTD peptide to date, it also possessed the similar, potent cell-penetrating activity. This finding is of great importance to medical research areas related to the use of PTD for achieving intracellular drug delivery, simply because LMWP owns several unmatched advantages over all of the existing PTD peptides. First, unlike other PTDs that rely solely on chemical synthesis for their production, LMWP can be manufactured in mass quantities using enzymatic hydrolysis and an established single step purification system. Secondly, unlike most other PTDs which are derived from viral resources and thus present health concerns, LMWP is obtained from digestion of native protamine, a FDA approved clinical drug. Thirdly, unlike all existing PTDs, the toxicology profile of LMWP has already been thoroughly established; as previously presented. Last but not least, since LMWP possesses only one single —NH₂ group at the N-terminus, its conjugation to a protein drug can be precisely regulated and easily carried out using the standard and well-established N-succinimidyl-3-(2-pyridyldithio) propionate (SPDP) activation method.

The concept of the proposed encapsulation technology was fostered primarily from phenomena observed from the aforementioned cell-penetrating, PTD peptides. In their animal study, Schwarze and coworkers reported that upon intraperitonial administration, the fusion protein of TAT (the most widely studied PTD) and □-galactosidase, with an overall MW >100 kDa, was effectively but non-selectively transduced into every organ and tissue, including kidney, heart and even the brain. This finding suggested that intracellular protein uptake mediated by the PTD peptide was not receptor- or transporter-dependent, because it was not possible that all different types of cells would possess the same types of receptors or transporters. Based on this conclusion, in principle all cell types including erythrocytes should be transducible. In addition, since it was also demonstrated that PTD-mediated cell internalization did not induce any perturbation or alteration of the cell membrane, these PTD peptides could potentially be applied as a powerful tool to achieve non-invasive encapsulation of biologically active protein therapeutics into intact and fully functional erythrocytes. These hypotheses provide the framework of our proposed innovative method for encapsulation of ASNase into RBCs.

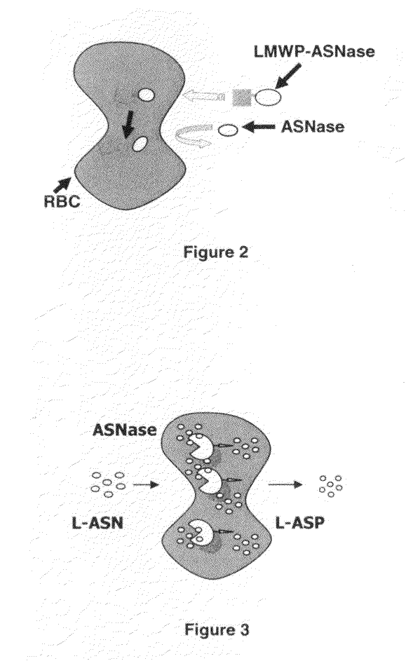

As displayed in FIG. 2, the new encapsulation method calls for covalent conjugation of ASNase with a PTD peptide via disulfide linkages. Because of the universal and potent membrane-penetrating activity of PTD, even without the involvement of any invasive membrane-disrupting procedures, the PTD-ASNase conjugates should still be able to internalize erythrocytes without altering RBC's structural and/or functional attributes. Within the cells, PTD would be by design dissociated from its protein counterpart via degradation of the disulfide linkage, due to the presence of a high level of cytoplasmic glutathione and reductase activities. This bond dissociation would enable the cell-impermeable ASNase to remain permanently entrapped in the erythrocytes, ensuring protection of the enzyme drug from detection by the host immune system and clearance by RES and other endogenous factors.

Ataullakhanov and co-workers demonstrated that the substrate asparagine was able to permeate into human erythrocytes from external medium. Hence, ASNase-encapsulated erythrocytes would function as a living bioreactor, depleting ASN in the circulation and depriving leukemic cells of their essential nutrient and, consequently, leading to death of these cells (see FIG. 3). If physical and biological attributes of erythrocyte can be completely maintained following the encapsulation process, then the RBC-entrapped ASNase would presumably possess the same circulating life-span of native erythrocytes (i.e. 120 days), yielding longer anti-cancer therapeutic effect than that of ASNase delivered by any other synthetic or natural drug carriers.

L-Asparaginase (ASNase) is an enzyme drug that has been used routinely in clinical practice for the induction of remission in patients with acute lymphoblastic leukemia (ALL). Despite wide use, its clinical applications face two major shortcomings. First, like most protein compounds, ASNase is highly susceptible to degradation by circulating serum proteases and/or clearance by the host immune surveillance system. Secondly, systemic exposure to ASNase can result in manifestation of immunological responses including activation of B lymphocytes and production of antibodies, leading to severe and, at times, fatal anaphylactic reactions. Because of the unmatched circulating life-span (˜120 days), erythrocytes have been investigated extensively to be used as a drug carrier in protecting ASNase from proteolytic digestion and elimination by the reticuloendothelial system. Yet, existing RBC encapsulation technologies, which all involve disruption of the cell membrane with either chemical, electrical, or physical forces to create large pores for the drug diffusion, would inevitably result in an irreversible deterioration of the structural and morphological integrity of, as well as a loss of important cellular components of the erythrocytes. As a consequence, thus treated RBCs appear pink in color and have an abnormal, spherical or stomatocyte shape (i.e. oval or rectangular area of central pallor; see FIG. 1 above) with markedly weakened surface structures. Widely termed as “Ghost-RBCs”, they are recognized by the host body as foreign entities and will therefore be rapidly cleared by the phagocytic system. To this regard, the direct significance of the described RBC encapsulation technology lies in its ability to completely preserve all of the benefits of RBC protection, thereby offering the highest degree of enhancement in ASNase therapy. As far as our knowledge goes, the proposed application of the membrane-penetrating PTD peptides (e.g. LMWP) for intracellular protein transduction provides the first methodology to allow for non-invasive encapsulation of therapeutically active protein drugs into both structurally and functionally intact erythrocytes. Preliminary results showed that erythrocytes treated by this method not only exhibited a long plasma half-life similar to that of untreated RBCs, but also displayed enhanced therapeutic effects by the entrapped ASNase in mice harboring leukemia cells; presumably via protection of ASNase from possible proteolytic degradation and phagocytic clearance. This would permit the use of a dramatically reduced dosing frequency (i.e. by more than 100 fold) to achieve the same therapeutic efficacy over a long period of time interval; thereby significantly reducing the toxic effects associated with current ASNase therapy. In addition, a full preservation of the intact structure and functionality of the processed RBCs is also of great significance, because in theory it would provide the flexibility of replacing an unrestricted amount of blood (or RBCs) from the patient with drug-loaded erythrocytes; should situations warrant such a clinical management.

Also of significance is the simplicity and practicality of this system for potential clinical uses. An advantage of RBCs is that they completely biodegrade without the generation of toxic byproducts. In addition, compared with other cargo systems, RBCs are clearly by far the most compatible drug carriers particularly when autologous species are used. On the other hand, presently ASNase is produced via the recombinant method. Since LMWP is a peptide compound, in principal and practice, the LMWP-ASNase conjugates could be readily and similarly prepared without altering much of the manufacturing process or costs. Ample examples of success with regard to the synthesis of a great variety of PTD-protein conjugates using the recombinant method have been reported in literature. Moreover, since LMWP is derived from a clinically approved drug (i.e. protamine) and yet with a significantly reduced toxicity than the original protamine, its involvement in the ASNase conjugates should not raise safety concerns. Since the cell internalization process mediated by LMWP is extremely efficient, and since this process is self-initiated (i.e. receptor-independent and also without the aid of a chemical or physical force) and temperature-independent, it is envisioned that encapsulation, of ASNase into RBCs could be swiftly accomplished by infusion of the packed RBCs, collected from a routine clinical blood separation procedure, into a blood collection bag containing sterilized LMWP-ASNase solution at 4° C. After 1-2 hrs of incubation in the cold, the ASNase-loaded RBCs could then be infused back to the same patient; a procedure closely mimicking the clinical situation of a delayed blood transfusion process. Overall, the entire ASNase-loaded RBC system could virtually be constructed with FDA-approved components and GMP-regulated processes, rendering it suitable for clinical applications.

The primary reason of selecting ASNase as the primary drug target for validation of this new RBC encapsulation technology was because ASNase had been attempted in almost every RBC encapsulation techniques, and therefore a direct comparison of the benefits of this new method over existing ones could easily be made and assessed. It should be noted that because of their unmatched substrate specificity and reaction efficiency, proteins or gene products have recently been recognized as the new trend of therapeutics. Indeed, a large number of such macromolecular compounds including hormones, antibodies, vaccines, cytokines, enzymes, proteases, DNAs and siRNAs have been identified as effective therapeutic agents, and are currently either under development or in clinical trials. Because of the universal capability of the PTD peptide to effectively translocate all types of macromolecules into all cell types, it is quite possible that the presented technology could evolve as a generic approach to encapsulate a wide range of macromolecular drugs into a great variety of living cells for protein or gene therapy, or even for tissue engineering. Indeed, encapsulation of several other protein drugs into live cells for treatment of neurodegenerative disorders (e.g. Alzheimer and Parkinson diseases), cocaine overdose, and various types of cancers are currently being pursued by ISTN scientists and Professor Yang's research group. Therefore, the significance and impact of this new encapsulation method is far-reaching, prevalent, and wide-spread.

Based on its past experience and success in translating SBIR research to commercial products, ISTN has already established a three-phase plan to commercialize the proposed RBC-encapsulated ASNase technology. The specific aims of Phase I research have already been discussed in detail in Section A. Briefly, during Phase I work, the main focus will be to carry out proof-of-concept animal studies to completely validate the plausibility of this RBC-encapsulation technology. The four specific aims related to this phase of research will include: [1] further evaluation of the pharmacokinetic profiles of both normal RBCs and ASNase-loaded RBCs; [2] assessment of the therapeutic benefits of the RBC-loaded ASNase; [3] testing of the immunogenicity of the RBC-ASNase; and [4] examination of the toxicity of the RBC-ASNase system.

Once the feasibility of this RBC-ASNase system is confirmed, Phase II work will be geared towards translating this technology into a real clinical remedy. Since the final product involves labile and delicate blood components with restricted storage time (it has been reported that RBCs can be stored under refrigeration for up to 42 days), it is envisioned that ASNase-loaded erythrocytes can not possibly be manufactured as a commercial product, but instead be processed in a service center in an on-demand, time-sensitive fashion. To this regard, establishing a GMP-regulated protocol for processing the entire RBC encapsulation event becomes inevitable. Thus, the chemical procedures employed in synthesis of the LMWP-ASNase conjugates simply can not meet with these GMP requirements. Hence, during Phase II, we plan to establish a recombinant method that can produce the LMWP-ASNase conjugates in mass quantity, similar to current manufacturing process of preparing clinical ASNase product. Further, we also plan to establish a GMP-approved protocol for preparing the RBC-encapsulated ASNase, such as the procedures discussed previously by infusion of the packed RBCs (i.e. collected from a routine clinical blood separation procedure) into a blood collection bag containing sterilized LMWP-ASNase solution at 4° C., and after 1-2 hrs of incubation in the cold, the ASNase-loaded RBCs is then infused back to the same patient; a process mimicking a delayed clinical blood transfusion procedure. Once these GMP-based manufacturing processes are established, extensive animal studies related to the efficacy, safety, pharmacokinetics, toxicology etc., will be carried out by using these RBC-ASNase products, to further assess the possibility of developing this technology into a real clinical remedy.

During a later stage of the Phase II work, ISTN will begin to initiate contact with potential licensing partners. In Phase III, ISTN will attempt to transfer the established technology to selected partners with the goal to establish a service center-like network to commercialize the technology. The partner company is expected to work closely with ISTN in carrying out the clinical trials and marketing the new product upon FDA approval.

### Preliminary Studies

Extremely promising results have been attained through several key preliminary studies. In general, these investigation were focused on demonstrating the feasibility of the proposed research on two fronts: [1] determine if the low molecular weight protamine (LMWP) peptide possesses a potent cell-penetrating activity similar to that of TAT; the most widely studied PTD to date; and [2] applying the LMWP-mediated cell internalization technology, whether ASNase can be non-invasively encapsulated into structurally intact, completely functional RBCs without altering any of the chemical/physical attributes of such erythrocytes.

### A. LMWP Functions as a Potent PTD Peptide

The main benefits of the non-toxic LMWP peptide have been discussed earlier in Section B.2 and will not be reiterated here. In general, LMWP was prepared by enzymatic digestion of protamine with thermolysin, followed by purification using a heparin affinity column. Upon elution, five peptide fragments, termed as TDSP1-5 (Thermolysin Digested Segment of Protamine 1 to 5) according to an increasing affinity for heparin, were obtained. TDSP5 with the highest arginine content was designated as LMWP. As seen, this peptide bears structural similarity to many of the arginine-rich PTD peptides, such as the most widely studied HIV-TAT peptide (Table 1). Because of this similarity, it was of great interest to examine if LMWP would possess the similar potent cell-penetrating activity.

TABLE 1
Amino Acid Sequences for TAT and LMWP

| | Amino Acid Sequence |
|---|---|
| HIV-TAT₄₇₋₅₇ | GRKKRRQRRRPPQ |
| LMWP(TDSP5) | VSRRRRRRGGRRRR |

### B. Cell-Penetrating Activity of LMWP

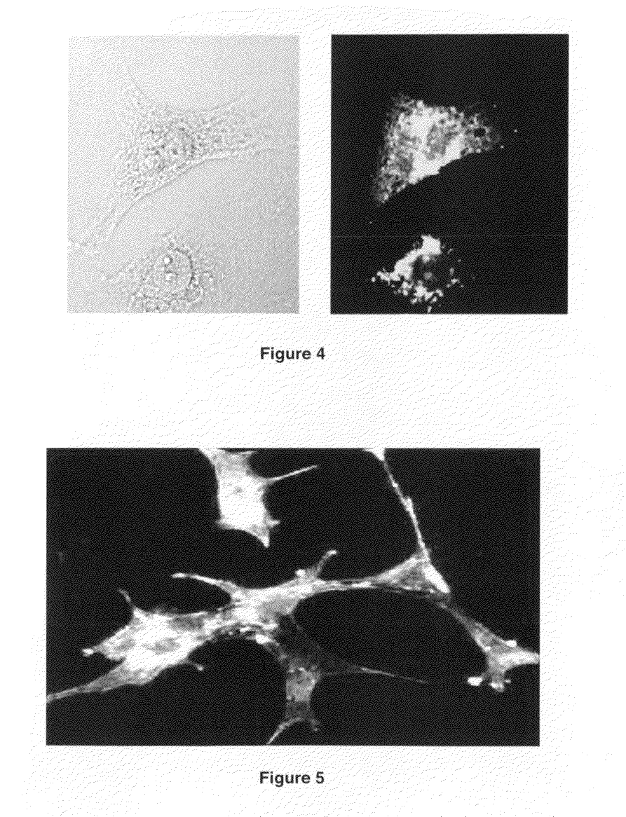

To investigate the membrane-penetrating activity of LMWP, cells were incubated with fluoroscein isothiocyanate (FITC)-labeled LMWP for 30 min. As seen in FIG. 4, LMWP displayed a significant cellular uptake, comparable to that of TAT (data not shown). Strong green fluorescence could be observed within the cells that had been exposed to LMWP-FITC and was clearly distinct from cell surface attachment.

### C. In Vitro LMWP-Mediated Cellular Uptake

The main characteristic of a PTD peptide lies in its ability to carry large, cell impermeable cargos across the cell membrane and into cytosol. To confirm this ability, LMWP was linked to rhodamine-labelled phalloidin, a cell-impermeable labeling agent that binds actin filaments of the cell cytoskeleton. While there was no intracellular uptake of free phalloidin after incubation with MG63 osteroblast cells (data not shown), FIG. 5 revealed that phalloidin-LMWP conjugates were capable of internalizing cells, as reflected by the clear cytoskeletal labeling, without any disruption of the cell membrane structure.

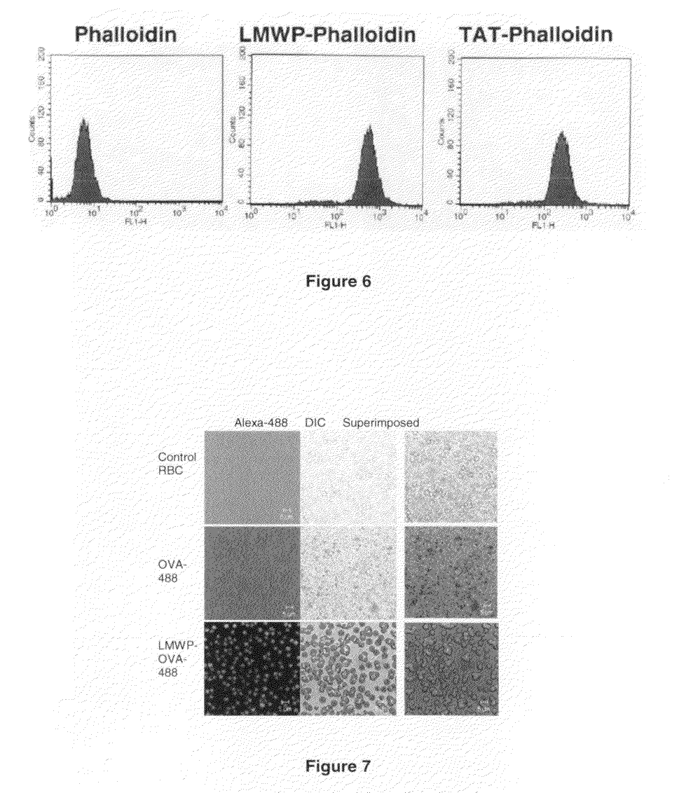

Flow cytometric analysis yielded similar results. As shown in FIG. 6, while there was virtually no intracellular fluorescence for Alexa Fluor 488-labeled free phalloidin, a strong cellular uptake was observed for fluorescence-labeled phalloidin-LMWP conjugates. Interestingly, both phalloidin-LMWP and phalloidin-TAT conjugates displayed almost identical intracellular uptake, suggesting that cell-penetrating activity for LWMP and TAT was comparable.

### D. RBC Encapsulation of Protein Drugs

Ovalbumin as the Testing Protein

To examine the general feasibility of LMWP to translocate proteins into erythrocytes, we first adopted Alexa Fluor 488 (a fluorescent dye)-labeled ovalbumin as the protein model. Briefly, LMWP was introduced with a thiol (—SH) functional group at its N-terminus by using the bifunctional cross-linker 3-(2-pyridyldithio)propionic acid N-hydroxysuccinimide (SPDP) activating agent, and was then coupled with a similarly SPDP-activated ovalbumin molecule. As shown in the confocal microscopic images in FIG. 7, RBCs incubated with the dye-labeled ovalbumin displayed only vague auto-fluorescence from hemoglobin excitation on the cell surface, with no observable uptake of the labeled protein within the interior of the cells (FIG. 7b); a phenomenon that was almost identical to that of the untreated control RBCs (FIG. 7a). On the contrary, after conjugation with LMWP, significant intracellular fluorescence was detected within the RBC carriers, confirming the occurrence of LMWP-mediated cell internalization of the otherwise impermeable ovalbumin (FIG. 7c).

RBC Encapsulation of L-Asparaginase

As suggested by the above findings, LMWP was capable of transporting large protein cargos (e.g. ovalbumin) into RBCs. To confirm the utility of this non-invasive encapsulation method for protein drugs, asparaginase (ASNase), an enzyme drug employed clinically in the treatment of acute lymphoblastic leukemia (ALL), was activated with SPDP and then coupled with SPDP-activated LMWP via a cytosol-degradable disulfide linkage, according to the same procedures described herein.

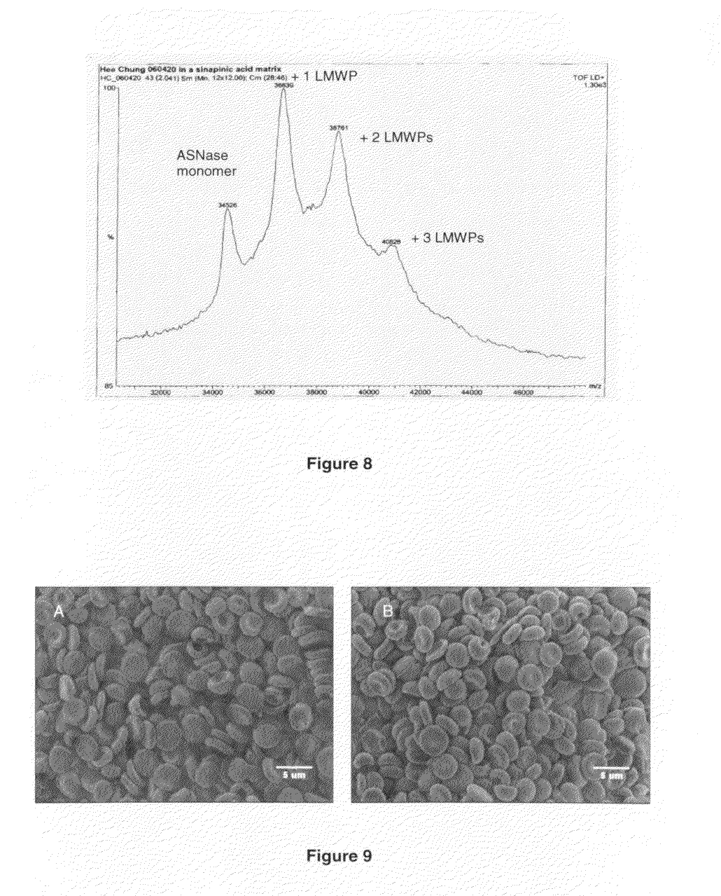

The MALDI-TOF mass spectra shown in FIG. 8 revealed that 1-4 LMWP peptides were conjugated to each L-asparaginase monomer. The specific activity of ASNase was found to be approximately 60% of the native enzyme, after conjugation.

For encapsulation, RBCs were incubated with the LMWP-ASNase conjugates at a total ASNase concentration of 100 IU/mL. A loading efficiency of 4% was observed, with a loading capacity of 8 IU ASNase per packed volume of 100 μL of packed RBCs. During the loading process, no hemolysis or loss of hemoglobin was detected. In addition, no leaching of ASNase from the loaded RBCs was detected during the first 14 days of incubation in isotonic buffer at 4° C. After this period, both control and ASNase-loaded RBCs began to show signs of disintegration in vitro. When the loaded RBCs were lysed after 14 days of incubation, 70% of ASNase activity could be recovered. Since the RBCs were treated with trypsin and washed with Alsever's solution after ASNase loading, the recovered enzymatic activity was clearly from the intracellularly entrapped ASNase. It should be noted that in clinical practice, the dose regimen for ASNase as a sole induction agent in ALL treatment is about 200 IU/kg body weight. Hence, even based on our currently established loading protocol (i.e. 8 IU ASNase per 100 μL of packed RBCs), this clinical dosing regimen, which can be translated into a dose of 3 mL of ASNase-loaded RBCs per kg of the patient's body weight, is obviously achievable.

It was speculated that the detachment of LMWP from ASNase via degradation of the disulfide bonds by the elevated glutathione activity in the cytosol caused the membrane-impermeable ASNase to be trapped inside. On the other hand, so far there has been no report to implicate that the PTD-mediated cell entry is a reversible process. Hence, the permanent entrapment of the protein drug inside RBCs could also result from this irreversible translocation process. Albeit controversial, most literature reports suggested that the final destiny of the PTD-mediate event was the nucleus. Since RBCs are non-dividing and non-nucleated cells, it is without any doubt that the entrapped ASNase stay in the cytosol of the RBC. Overall, the absence of leaching and activity decay of the entrapped ASNase indeed fulfills one of the essential prerequisites for the ASNase-loaded RBCs to be considered as a real clinical remedy.

Morphological Integrity of RBCs after Encapsulation with LMWP-ASNase

Since conventional RBC-encapsulation methods such as electroporation or hypo-osmosis-based techniques all inevitably involve perturbation of the RBC plasma membrane during drug entrapment, the surface morphology of the ASNase-loaded RBCs was examined. Unlike the enzyme-loaded RBCs created using the other methods, which displayed changes in surface morphology to the abnormal stomatocyte form (see FIG. 1 above), FIG. 9 showed that RBCs treated with LMWP-ASNase (FIG. 9B) exhibited a surface morphology that was indistinct from the native erythrocytes (FIG. 9A), with the preservation of the customary biconcave shape and no observable deformities or perforations.

Structural and Functional Integrity of ASNase-Encapsulated RBCs

As discussed previously, because of the physical, chemical, and mechanical insult applied to the cell membrane to create diffusible pore structures for protein drugs, RBC processed by any of the existing encapsulation techniques would inevitably result in a loss of both the structural and functional integrity of the processed erythrocytes. Numerous reports have been documented in the literature to indicate that RBCs treated by such methods were always accompanied with altered cell volumes and content of cellular constituents such as hemoglobin and cytoskelon. It should be reminded that a loss of hemoglobin would significantly impair the oxygen transport functions of the erythrocyte, whereas a loss of cytoskeleton would compromise the erythrocyte with a weakened structural integrity, rendering it prone to destruction and clearance by RES and the phagocytic system.

Structural Integrity of Treated RBCs

To assess the structural integrity of the ASNase-loaded RBCs, several key hematological parameters including the mean corpuscular volume (MCV), mean hemoglobin content (MHC), and mean corpuscular hemoglobin concentrations (MCHC) were examined. As seen in Table 2, all of the measured parameters of the ASNase-loaded RBCs were virtually statistically indistinguishable from those obtained for the untreated RBCs (i.e. Control).

TABLE 2
Hematological Parameters.
Measurements were performed according to procedures described below.

| | Control (sd) | Loaded (sd) |
|---|---|---|
| MCV (fL) | 32.63 (0.06) | 32.53 (0.06) |
| MCH (pg) | 12.37 (0.50) | 12.23 (0.45) |
| MCH (g/dL) | 37.97 (1.62) | 37.60 (1.45) |

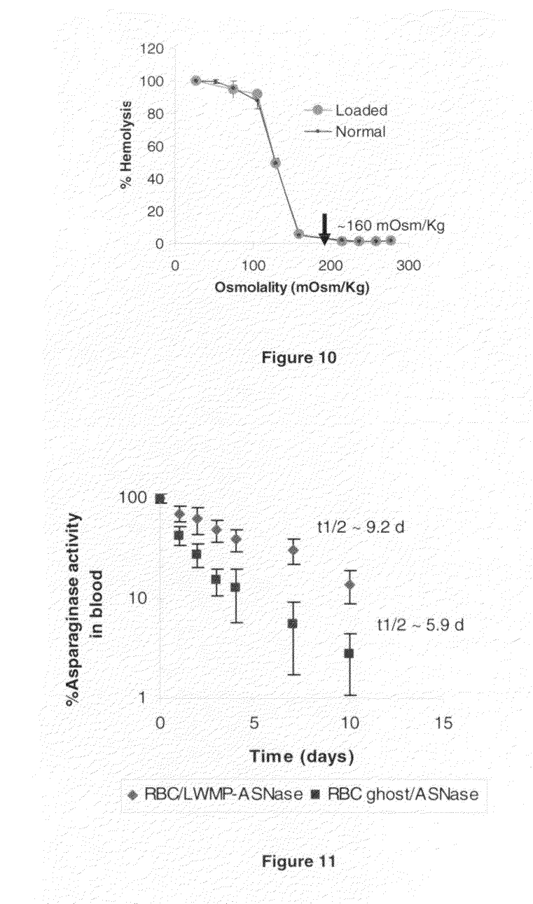

As discussed previously, all of the existing methods involved in incorporation of protein drugs into RBCs would more or less require a certain degree of membrane perturbation or disruption. To this regard, osmotic fragility of the treated RBCs could be used as a definite measure or a revealing sign of the membrane integrity following the drug loading process. Numerous reports have been found in the literature to indicate changes in osmotic fragility curves after loading erythrocytes with the hypotonic methods. In a comparison study of erythrocytes prepared by two of the most promising encapsulation methods, hypotonic dialysis/hypotonic wash versus hypotonic dialysis/isotonic wash, Chiarantini and co-workers demonstrated that both samples exhibited distinctly different osmotic fragility profiles from normal RBCs, as well as markedly higher rupture osmolality values compared to the normal erythrocyte. These findings signified the view that the RBC membrane was considerably weakened after drug loading via such methods. In sharp contrast, our results in FIG. 10 showed that erythrocytes processed with the LMWP-ASNase loading method displayed a nearly superimposed osmotic fragility profile to that of normal erythrocytes, with both samples showing an identical onset of rupture osmolarity of about 150 mOsm. These findings further validate our hypothesis that LMWP-mediated ASNase encapsulation was a relatively non-invasive process that did not compromise the RBC membrane with a weakened structural integrity.

Functional Integrity of Treated RBCs

As noted earlier, during RBC processing with the conventional pore-forming or osmotic-swelling protein encapsulation technique, a loss of important plasma components from the treated erythrocyte, specifically hemoglobin, becomes inevitable. As a consequence, a change of the normal oxygen-carrying capability of the drug-loaded erythrocytes is fully expected. Nevertheless, when subjected to varying oxygen concentrations, it was found that the hemoglobin from the ASNase-loaded RBCs yielded the same characteristic sigmoidal curve indicative of cooperative binding as that of the native RBCs. Most importantly, the Hill coefficient and pO₅₀ values obtained for the ASNase-loaded erythrocytes, which represented the functionality of hemoglobin in the erythrocyte, were statistically indistinguishable from the results, either from literature report or from our own experimental findings, of the untreated normal sheep erythrocytes (see Table 3).

TABLE 3
Oxygen-carrying functionality of ASNase-loaded sheep erythrocytes.
Hill coefficients and pO₅₀ values were measured according
to procedures described below.

| | | | Literature Data |
|---|---|---|---|
| | Control | Loaded | (sheep RBC) ** |
| Hill Coefficient | 3.15 ± 0.32 | 3.07 ± 0.34 | 3.14 ± 0.12 |
| pO50 (mmHg) | 37.44 ± 0.03 | 38.64 ± 0.02 | 40.0 ± 1.0 |

Prolonged Circulating Half-Life of ASNase-Loaded RBCs

The true proof of the benefits of the LMWP-mediated cell encapsulation method stemmed from the in vivo pharmacokinetic investigation of the plasma half-life of the ASNase-loaded erythrocytes. To conduct the experiments, ASNase-loaded RBCs were injected into the tail vein of DBA/2 mice, and the circulating half-life was determined based on the linear portion of the elimination phase. For comparison, RBCs loaded with ASNase via the standard hypotonic method were used as a control. Consistent with findings by other investigators the hypotonic method resulted in changes in morphology and surface structures of many of the processed erythrocytes and, as a result, markedly shortened the circulating half-lives of such cells. As shown in FIG. 11, a half-life of approximately 5.9 days was found for ASNase-loaded erythrocytes processed with the hypotonic method; which was well within the range between 4.5 to 7.8 days reported by other investigators. In sharp contrast, ASNase-loaded erythrocytes processed with the LMWP-mediated encapsulation method exhibited a significant prolonged plasma half-life of 9.2 days; almost doubled the value obtained for the hypotonic-treated erythrocytes. A number of reports of the half-lives of normal mouse erythrocytes, generally in the range between 5.3 and 9.5 days, were documented in the literature. Yet, since all these studies involved extraction of erythrocytes from the animal, processing such cells with ⁵¹Cr-labeling, and then re-injected these labeled RBCS back into the mice for the pharmacokinetic studies, the half-lives acquired from such studies therefore did not represent the precise and actual half-life of the mouse erythrocytes. In addition, these data were also relatively inconsistent pending on the conditions and techniques employed during RBC processing. Hence, it was not conclusive of whether the half-life of 9.2 days obtained from our pharmacokinetic study was the actual half-life of normal mouse erythrocytes. However, because this data approximated the high-end extreme of the half-life range (i.e. between 5.3 to 9.5 days) obtained by other investigators for normal mouse erythrocytes, it would be reasonable to assume that erythrocytes processed by this new encapsulation method behaved equivalently, or at least very closely to the same physiological manners of untreated RBCs. More conclusive studies to verify this assumption by utilizing ⁵¹ Cr-labeled erythrocytes are proposed below.

Therapeutic Efficacy of ASNase-RBCs

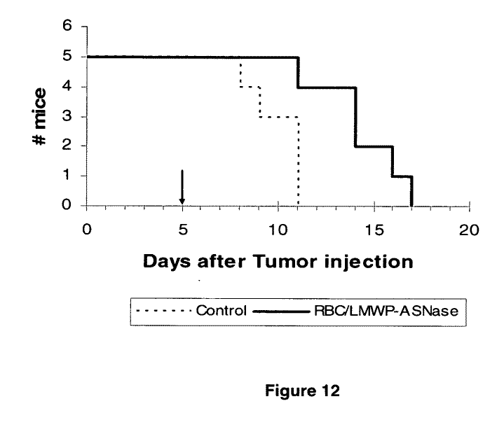

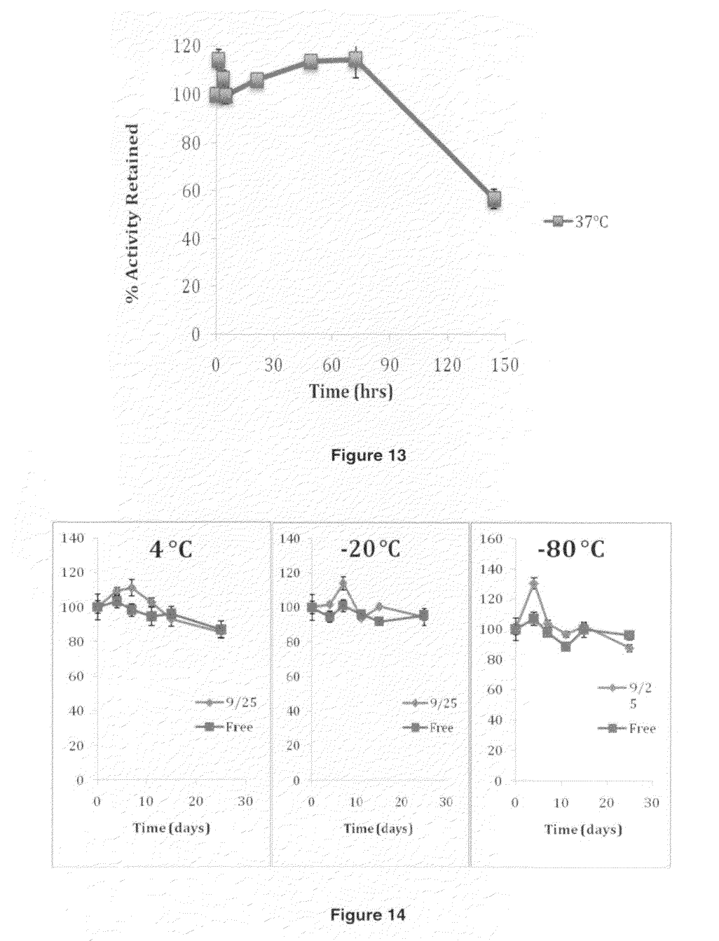

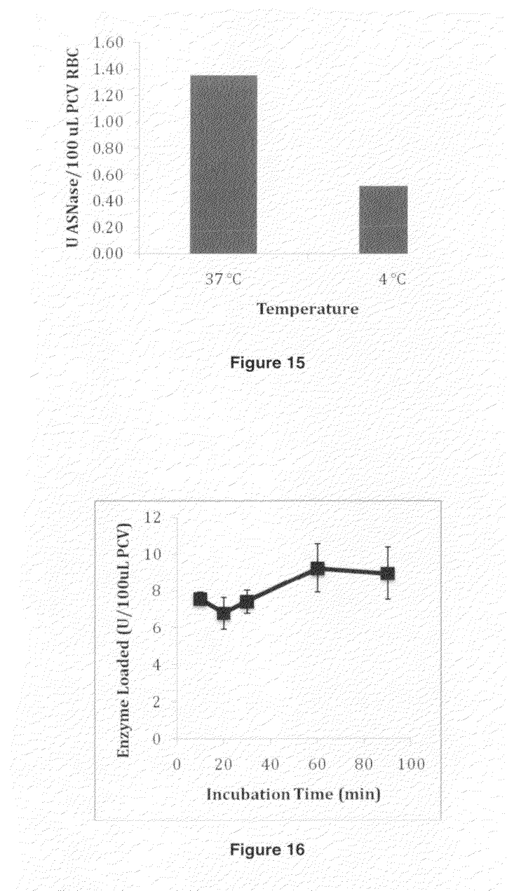

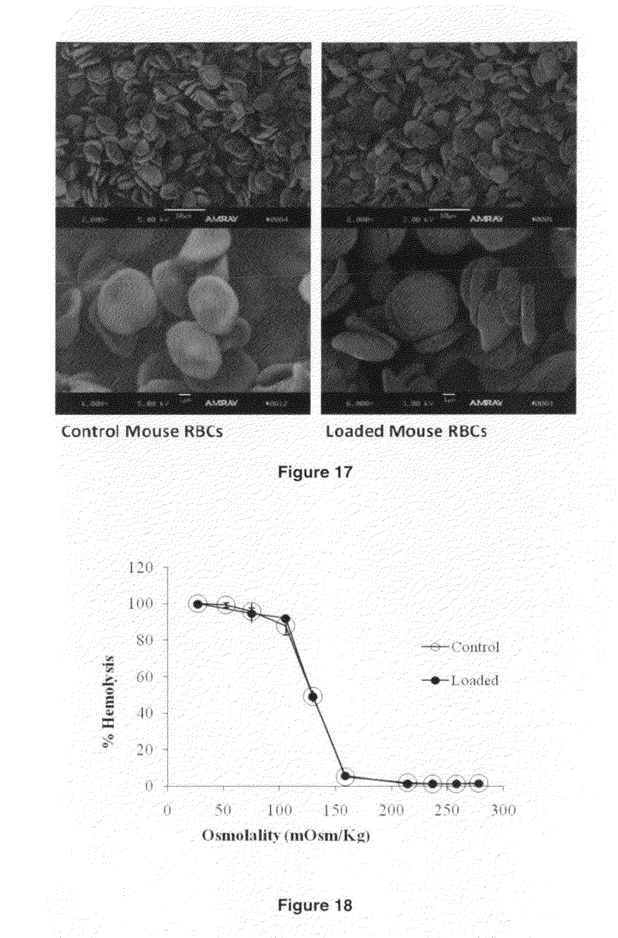

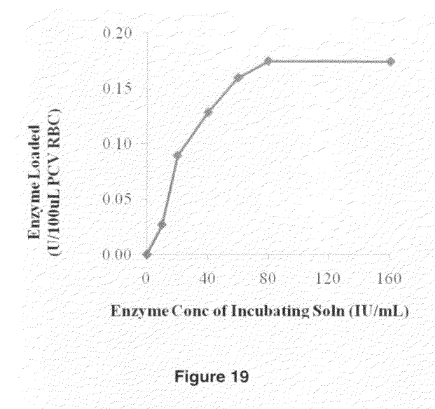

For the planned RBC-encapsulated ASNase technology to function desirably under clinical settings, another essential pre-requisite is that the entrapped ASNase must be able to retain its original therapeutic capability. To verify this key element, a preliminary animal study was carried out. In this proof-of-concept feasibility study, the therapeutic functions of the ASNase-loaded erythrocytes against the DBA/2 mice harboring the L5178Y lymphoma tumor cells were examined. The L5178Y tumor cell line was selected because it was documented in the literature to be highly sensitive to ASNase therapy, whereas the DBA/2 mouse model was chosen because L5178Y cells were known to be tumorgenic in this mouse strain. Five days after intraperitonial tumor implantation and when mice showed obvious visual signs of the tumor burden, 100 □L of saline solution (control) or ASNase-loaded erythrocytes containing 8 IU ASNase activity (experimental) was administered intravenously to the mice. As can be seen in FIG. 12, administration of ASNase-loaded erythrocytes was able to considerably increase the survival time of the mice (14.4±2.3 days), when compared to the survival time of 10.0±1.4 days observed for the control, saline-injected animal group; a significant enhancement of the survival time by 44%.

Though the results were quite preliminary animal results (N=3), it was nevertheless quite easy to assess the prowess and clinical potential of the proposed technology on ASNase therapy, should a comparison be made to relate our results with findings by others under similar in vivo conditions. A maximum enhancement of 16.7% in the survival time over the control group was reported by other investigators after intravenous injection to the L5178Y tumor-bearing mice of a total dose of 8 IU of free ASNase. Yet, a nearly three-fold increase over their results (i.e. 44% versus 16.7%) in the enhancement of life span was observed for our RBC-ASNase treated mice, particularly considering the fact that merely a volume as small as 100 □L of the ASNase-encapsulated RBCs were infused into these tumor-bearing mice. Further studies to more conclusively demonstrate the therapeutic benefits of the proposed system by infusion of a larger volume (e.g. 300 □L) of the RBC-encapsulated ASNase are proposed below.

### E. Experimental Design

LMWP-ASNase Conjugate Synthesis

Low molecular weight protamine (LMWP) will be prepared according to previously described procedures. To activate LWMP, the N-terminal NH₂ group will be thiolated using the bifunctional cross-linker 3-(2-pyridyldithio)propionic acid N-hydroxysuccinimide (SPDP). Briefly, 5 mg/ml LMWP in 50 mM HEPES buffer (pH 8) will be mixed with SPDP (1:10 molar ratio) in DMSO and shaken for 1 h at room temperature. The reaction mixture will then be treated with 50 mM dithiothreitol (DTT), and the thiolated LMWP will then be purified by HPLC on a heparin affinity column. The final product will be collected by ultrafiltration, lyophilized, and then stored at −20° C. until further use.

For conjugation, 5 mg/mL L-asparaginase (238 IU/mg) will be mixed with SPDP (40 μL of 0.1 M SPDP in ethanol to 1 mL protein solution) in phosphate buffer, and stirred at room temperature for 1 h. Unreacted SPDP will be removed by rapid desalting and buffer exchange by FPLC with 0.1 M acetate buffer (pH 4.5). Activated ASNase will then be conjugated with a 10-fold molar excess of the previously prepared LMWP-SH for 24 h at 4° C., according to a previously established protocol. LMWP-ASNase conjugates will then be isolated by ion-exchange chromatography using a heparin affinity column followed by five rounds of centrifugal filtration (MW cut-off: 5,000 Da). Pooled LMWP-ASNase conjugates will be concentrated, and the degree of conjugation will be determined by MALDI-TOF mass spectroscopy.

RBC Encapsulation of LMWP-ASNase

Erythrocytes collected from mouse species will be suspended in HBSS at a density of 5×10⁸ cells/mL and centrifuged. Equal volume of 250 μg/mL of LMWP-ASNase in HBSS will then be added to the packed RBCs and incubated at 4° C. overnight. The cells will then be washed with Alsever's solution and collected by centrifugation at 800×g for 3 min. To remove the surface bound LMWP-ASNase conjugates, the cells will be treated with trypsin-EDTA for 5 min at 37° C. and washed twice with Alsever's solution. The degree of ASNase loading will be estimated by determining the ASNase activity via direct nesslerization of produced ammonia, according to previously described procedures.

Quality Control for the RBC-ASNase Products

Results presented previously in Section C.2.3 & C.2.4 have already confirmed that RBCs processed by the proposed LMWP-mediated encapsulation technology remain both structurally and functionally intact. To ensure an appropriate quality control of the products required for subsequent animal studies, several key parameters will be monitored closely from samples collected from the above-prepared RBC-ASNase products prior to their use in the in vivo experimentations.

Scanning Electron Microscopy

One hundred microliters of packed RBC-ASNase samples will be placed in E-well plates containing glass coverslips and incubated in 1% glutaraldehyde for 15 min at room temperature. The coverslips containing the fixed RBCs will be air-dried, gold-sputtered for 1 min, and then observed by scanning electron microscopy (1000-B SEM, Amray) at an accelerating voltage of 15-20 kV.

Unless otherwise stated, untreated RBCs will be used in this and also following two sections for comparison purposes.

Osmotic Fragility Measurements

Normal or LMWP-ASNase loaded erythrocyte samples will be suspended at concentrations of 50% hematocrit, and 20 μL of the suspended cells will be added to 1.0 mL solutions of hypotonic saline with increasing osmolality ranging from 0-300 mOsm/kg. The solutions will be incubated at 37° C. for 30 min, centrifuged and the absorbance of each supernatant be measured at 540 nm. The absorbance for 0 mOsm/kg solution will be taken as 100% hemolysis. The osmolality of each solution will be measured using a Wescor osmometer (Logan, Utah).

Hematological Parameters

A 10% solution of normal or ASNase-loaded erythrocyte samples in HBSS will be washed three times and lysed with distilled water. The resulting hemolysate will be centrifuged and the supernatant diluted to 5 mL with a 1:1 mixture of HBSS and distilled water. Dissolved oxygen will be measured using a Clark electrode (World Precision Instruments, Sarasota, Fla.) connected to a data acquisition system according to a previously established protocol. Oxyhemoglobin will be measured using a spectrophotometer (UV 2501PC, Shimadzu, Columbia, Md.; thermostated at 37° C.) at 540 nm, and changes in oxyhemoglobin content of the hemolysate due to decreases in oxygen concentration will be monitored. The pO₅₀ values will be measured, and Hill plots will be constructed according to a previously established protocol.

To determine the mean corpuscular volume (MCV), mean corpuscular hemoglobin (MCH), and mean corpuscular hemoglobin content (MCHC), erythrocytes will be analyzed using a veterinary hematology system (Drew Scientific, Dallas, Tex.).

In Vivo Evaluation of the Feasibility of the Proposed RBC-ASNase Technology

The primary goal of this SBIR Phase I research is to provide a proof-of-concept demonstration of the benefits of the RBC-ASNase system in enhancing anti-leukemia drug therapy. To achieve this goal, feasibility of this system must be confirmed on four research fronts: [1] RBC-encapsulated ASNase should possess a circulating life span comparable with that of native erythrocytes; [2] a significantly prolonged therapeutic efficacy should be obtained for the RBC-encapsulated ASNase; [3] RBC encapsulation should be able to prevent the entrapped ASNase from inducing immunogenic responses to the host system; and [4] the use of RBC-encapsulated ASNase should be able to markedly reduce the toxic effects of native ASNase. The four sections of experiments proposed below are specifically designed to verify such feasibilities respectively.

In vivo evaluation of the proposed RBC-ASNase technology will be carried out in Professor Victor C. Yang's laboratory at the College of Pharmacy, the University of Michigan. All animal experiments will be performed in accordance with the Guide for Laboratory Animal Facilities and Care (NIH publication 85-23, revised 1985) and the guidelines of the Institutional Animal Care and Use Committee (IACUC) of the University of Michigan.

Pharmacokinetics of ASNase-Loaded RBCs

One of the featured benefits of the proposed LMWP-mediated encapsulation technology is that it would preserve a complete structural and functional integrity of the erythrocytes after processing, thereby providing the encapsulated ASNase a prolonged life-span similar to that of normal erythrocytes (i.e. ˜120 days). Previous findings presented in Section C.2.5 already partially confirmed this phenomenon, as RBCs processed by our method yielded one of the longest circulating half-lives (i.e. approximately 9.2 days) when comparing to literature findings (i.e. between 5.3 and 9.5 days) reported for normal erythrocytes. To further evaluate whether the ASNase-loaded RBCs indeed possess a half-life comparable to that of normal, untreated RBCs, both types of cells will be labeled with ⁵¹Cr and then compared of results of their in vivo circulating half-lives. A well established protocol by Garin and co-workers will be followed to carry out the pharmacokinetic study of ⁵¹Cr-labeled RBCs. In brief, 1.5 mL of untreated or ASNase-loaded RBCs will be mixed 0.15 mL of ⁵¹CrO₄Na₂ in isotonic saline solution (1 mCi/mL, Dupont Co.) and incubated for 30 min at 40° C. under constant agitation. After washing 5 times with PBS solutions, 0.2 mL of ⁵¹Cr-labeled RBCs (60% hematocrit) will be injected intravenously by cadual tail vein into the Swiss CD1 male mice (30-35 g). At various time intervals, blood will be drawn by retro-orbital puncture with heparinized Pasteur pipette from anesthetized mice to measure the radioactivity using a gamma counter. The PK parameters will be determined by using the KINFT nonlinear least-squares program by fitting the plasma TCA-precipitated radioactivity data to the equation A(t)=A₁e^{-k}₁^{t}+A₂e^{-k}₂^{t}; where A(t)=% ID/mL plasma and ID=injected dose. The area under the plasma concentration curve (AUC), the steady-state volume of distribution (Vss), plasma clearance, and the mean residence time (MRT) will be calculated from A1, A2, K1, and K2 as described by Gibaldi and Perrier.

Unless otherwise stated, during the course of this and following studies, blood from healthy animals will be used as source of RBCs for control (untreated) and ASNase-loaded experiments. Healthy mice will be placed under surgical plane of anesthesia using ketamine/xylazine and blood collected by cardiac puncture. Blood from one mouse yields sufficient RBCs for conducting approximately 3-4 sets of in vivo experiments. Number of total animals to be used is calculated based on this information.

A total of 12 Swiss CD1 mice will be involved in this proposed pharmacokinetic study; 2 mice will be used solely for the collection of RBCs for sample preparations, whereas 6 mice each will be used for the control and ASNase-loaded RBC experiments.

Therapeutic Efficacy of RBC-ASNase

Very promising results were observed during the preliminary in vivo therapeutic efficacy study of the RBC-ASNase products; as 44% enhancement in survival time was experienced in tumor-bearing DBA/2 mice following treatment with an injection of merely 100 □L of ASNase-encapsulated RBCs (see Section C.2.6). In this continuation study, we plan to magnify the therapeutic effects of the RBC-ASNase system, by attempting to increase the injection volume of the drug product to 300 □L. To achieve this goal, 100 uL of blood will be withdrawn via retro orbital sinus before the administration of 300 uL RBC-ASNase suspension at 50% hematocrit. This process resembles “fluid replacement”, a practice used when more than 10% of total blood volume is removed from animal and accepted in veterinary practice. Aside from this change, all the other experimental conditions of the previous studies will be retained. Briefly, 30 DBA/2 mice will be involved in this investigation. They will be anesthetized with isoflurane and inoculated, intraperitoneally, with 1×10⁶L5178 leukemia cells in 0.1 mL HBSS. Three days after tumor implantation and when animals show obvious visual sign of tumor progression, mice will be randomly divided into three groups (10 mice/group) including: Group [1]: control mice that will be injected (via tail vein) with saline solutions only; Group [2]: mice will be injected with ASNase-loaded RBCs; and Group [3]: mice will be injected with free ASNase in conjunction with sham-loaded RBCs. The total ASNase dose given to each animal will be equivalent to 8 IU. Disease progression will be monitored using protocol titled “Tumor Burden Scoring System” put forth by Unit for Laboratory Animal Medicine (ULAM) at University of Michigan. The body weight change and survival times will also be recorded for comparison. Upon death of animal, peritoneal lavage will be performed to collect and count number of tumor cells.

Immunogenicity of RBC-ASNase

The purpose of this study will be two-fold. The first objective is to compare the immunogenic response arising from injection of free ASNase as opposed to that of RBC-encapsulated ASNase, whereas the second objective is to determine the immunogenic response arising from use of RBCs themselves as the drug carrier. The ideal situation would be to use autologous blood for immunogenic response, especially for that arising from

RBCs that underwent loading process. However, due to blood volume constraints in mice as well as the necessity for multiple injections, an alternative is proposed herein to achieve sample consistency, by pooling blood from a group of bled mice and then handling RBC samples uniformly for processing and injection. It should be noted that prior to blood collection, mice will be placed under anesthesia using ketamine/xylazine, and blood will then be collected by cardiac puncture.

Eighty Balb/c mice will be involved in this study. They will be randomly divided into 4 groups (20 mice per group) consisting of Group [1]: control mice that will be treated with saline solutions only; Group [2]: mice will be treated with free ASNase (8 IU) in conjunction with sham-loaded RBCs; Group [3]: mice will be treated with sham-loaded RBCs only; and Group [4] mice will be treated with ASNase-loaded RBCs (with an equivalent ASNase dose of 8 IU). Animals will be immunized via tail vein injection on days 0, 10, 20, and challenged again on day 30. On days 9, 19, 29, and 39, 5 animals from each group will be randomly selected, their blood will be collected via cardiac puncture, and plasma IgG levels will be determined using the ELISA assay described by Baran et al. It should be noted that blood will be drawn from all of the animals prior to their immunization and used as the baseline control.

Toxicity Evaluation of RBC-ASNase

Thirty healthy Balb/c mice will be involved in toxicology evaluation. They will be randomly divided into three groups (10 mice/group) including: Group [1]: control mice that will be injected (via tail vein) with saline solutions only; Group [2]: mice will be injected with ASNase-loaded RBCs; and Group [3]: mice will be injected with free ASNase in conjunction with sham-loaded RBCs. The total ASNase dose given to each animal will be equivalent to 8 IU. Animals will be monitored using “Policy for End-Stage Illness and Humane Endpoints” published by ULAM at University of Michigan. The physical change resulting from therapeutic dose of ASNase for Group [2] (i.e. RBC-ASNase group) is expected to be less apparent compared to that of Group [3] (i.e. free ASNase plus sham RBCs). For this reason it is necessary to include another quantitative means to evaluate the toxic effects resulting from ASNase and/or RBC administration. To this regard, the liver function abnormalities will be examined as another sign of toxicity. Briefly, animals will be anesthetized with isoflurane and 100 uL blood will be drawn via tail vein. The plasma will be measured for change in aspartate aminotransferase (AST or SGOT), alanine transamidase (ALT), alkaline phosphatase (ALKP), and/or bilirubin level(s). Upon death, liver will be harvested and examined for signs of fatty changes. According to the literature both in human and mice studies the signs of liver toxicity are alleviated upon discontinuation of ASNase treatment. Since leukemic patients undergo ASNase treatment for extended period of time, the timing of blood collection and liver harvest in this study will be crucial in accurately assessing the actual liver toxicity situations.

### F. Human Subjects

Not applicable.

### G. Vertebrate Animals

1. Twelve Swiss CD1 mice, 30 DBA/2 mice, and 80 Balb/c mice will be involved in this Phase I feasibility studies. Animal studies will be contracted to Professor Victor C. Yang's research group at the College of Pharmacy, The University of Michigan.

2. Mice were selected for the therapeutic efficacy studies of this Phase I research because they were the most economic and commonly used species for such studies.

3. Animal studies will be performed at the University of Michigan. Animal husbandry will be provided by the staff of ULAM (Unit for Laboratory Animal Medicine) under the guidance of supervisors who are certified as Animal Technologists by the AALAS (American Association for Laboratory Animal Science). Both University of Michigan and SUNY-Buffalo are fully accredited by the AAALAC and the animal care and use program conforms to the standards in “The Guide for the Care and Use of Laboratory Animals”, DHEW pub. No. (NIH) 80-23, revised 1985.

4. Anesthetic agents will be used in the animal studies to minimize discomfort and pain to the animals. Upon notification of the SBIR Phase I award by NIH, a detailed application including the protocols and procedures of animal use will be submitted to and reviewed by the University Committee on Use and Care of Animals (UCUCA).

5. A detailed description of the method of euthanasia will be reviewed by UCUCA. The method will be consistent with recommendation of the Panel on Euthanasia of the American Veterinary Medical Association.

### Examples and Experimental Data

### A. Hematological Parameters

One good indicator of structural change in RBCs is their hematological values. Change in mean cell volume (MCV) means change in morphology of RBCs whereas decrease in mean cell hemoglobin (MCH) indicates extent to which RBC membranes were compromised so that hemoglobin from inside leaked out. As can be seen in tables below extensive change in MCV is observed for both hypotonic and electroporation methods as well as decrease in MCH. This morphological change can be verified through scanning electron microscope (SEM) images of RBCs (FIG. 1).

TABLE 4
Hematological parameters of mice and human RBCs encapsulated
by hypotonic method. MCV = mean cell volume, MCH =
mean cell hemoglobin, and MCHC = mean cell hemoglobin content.¹

| | Mice | | Human | |
|---|---|---|---|---|
| Parameters | Native | Loaded | Native | Loaded |
| MCV (fL) | 50 ± 1 | 48 ± 1 | 94 ± 2 | 79 ± 1 |
| MCH (pg) | 17.7 ± 1.0 | 16.1 ± 1.1 | 32.0 ± 0.8 | 25.6 ± 0.9 |
| MCHC (g/dL) | 33.0 ± 1.5 | 33.2 ± 1.7 | 34.2 ± 1.1 | 33.1 ± 0.7 |

¹Kravtzoff, et al. (1990) J. Pharm. Pharmacol. 42, 473-476.

TABLE 5
Hematological parameters of mice RBCs
encapsulated by electroporation.²

| Parameters | Native RBCs | Electroporated RBCs |
|---|---|---|
| MCV (fL) | 52.1 ± 0.5 | 71.9 ± 0.9 |
| MCH (pg) | 17.5 ± 0.4 | 15.1 ± 0.9 |
| MCHC (g/dL) | 33.6 ± 0.7 | 21.1 ± 1.4 |

²Lizano, C., Perez, T. M., Pinilla, M. (2001) Life Sciences. 68, 2001-2016.

### B. Methods

LMWP-ASNase Preparation

LMWP and ASNase (Elspar, Ovation Pharmaceuticals, Inc., Deerfield, Ill.) were conjugated via disulfide bridge. First, LMWP was activated with heterobifunctional cross-linker SPDP (Sigma-Aldrich, St. Louis, Mo.). A five-fold excess SPDP dissolved in DMSO was added drop wise to LMWP dissolved in 0.1 M phosphate buffer containing 1 mM EDTA, pH 8.0 and reacted at room temperature for 2 hours. Activated LMWP was purified by heparin affinity column and concentrated using ultrafiltration cell, MWCO 500. ASNase was dissolved in 0.1 M HEPES buffer containing 5 mM EDTA and 10 fold excess Traut's reagent (Sigma-Aldrich, St. Louis, Mo.) dissolved in same buffer was added. ASNase was reacted with Traut's reagent for 1 hour at room temperature and purified using desalting column. Finally to the thiolated ASNase, 5 fold excess activated LMWP (relative to thiol) was added and reacted at room temperature for 1 hour. The final product was then purified by heparin affinity column and ultrafiltration, MWCO 5000.

ASNase Activity Measurement

ASNase activity was determined by direct nesslerization of produced ammonia. Enzymatic activity unit is defined as μmol ammonia produced per minute. Specific activity of native ASNase ranged from 206 to 259 units/mg of protein.

LMWP-ASNase Stability

Solution of LMWP-ASNase at concentration of 50 ug/mL was prepared. Three aliquots of this solution was stored at 4° C. and another three at 37° C. At specific time points, 50 uL from each vial was collected and measured for enzyme activity.

LMWP-ASNase Storage Stability

Two hundred microliter aliquots of LMWP conjugated or free ASNase solutions (˜1 mg/mL each) were prepared and stored at 4, −20, and −80° C. At specified time points, one vial was removed from each storage location and diluted to 50 ug/mL before measuring for enzyme activity.

Blood Collection

The blood was collected from anesthetized mice by cardiac puncture and placed immediately into microcentrifuge tube containing EDTA as anticoagulant. Blood was centrifuged to remove serum and RBCs were washed three times with R-HBSS before use.

Effect of Temperature on Loading

RBCs were incubated in LMWP-ASNase in R-HBSS (20 uL PCV RBC/75 IU ASNase/mL) for 1 hour at 4° C. and also at 37° C. RBCs were then washed and lysed to measure for enzyme activity.

Loading Kinetics Experiment

Twenty microliters of packed cell volume (PCV) RBCs were added to vials containing 1 mL of ASNase-LMWP at 20 IU/mL and incubated in shaking water bath at 37° C. At specified time points, vials were removed and centrifuged to collect RBCs, which were subsequently washed three times with R-HBSS before lysing to analyze for enzyme activity. For each time point the RBCs incubated in R-HBSS only was used as blank.

Hematological Parameters

To determine the mean corpuscular volume (MCV), mean corpuscular hemoglobin (MCH), and mean corpuscular hemoglobin content (MCHC), RBCs resuspended in R-HBSS at 50% hematocrit were analyzed using a commercially available veterinary hematology system (Drew Scientific, Dallas, Tex.).

Oxygen Dissociation Measurement

A 10% solution of ASNase loaded RBCs in HBSS was washed three times and lysed with distilled water. The resulting hemolysate was centrifuged and the supernatant was diluted to 5 ml with a 1:1 mixture of HBSS and distilled water. Dissolved oxygen was measured using a Clark electrode (World Precision Instruments, Sarasota, Fla.) connected to a data acquisition system. Oxyhemoglobin was measured using a 37° C. thermostated spectrophotometer (UV 2501PC, Shimadzu, Columbia, Md.) at 540 nm, and change in oxyhemoglobin content of the hemolysate due to decreases in oxygen concentration was monitored.

SEM

RBCs were fixed in 2.5% glutaldehyde in R-HBSS for 1 hour at 4° C. and washed three times with R-HBSS. Cells underwent dehydration in graded ethanol starting from 50% and finally in absolute ethanol. Dehydrated RBCs underwent four washes with HMDS and air dried over night. After gold sputtering (Polaron E5100) cells were examined by scanning electron microscope (1910 Field Emission Scanning Electron Microscope, Amray).

Osmotic Fragility

LMWP-ASNase or sham loaded RBCs were resuspended to 50% hematocrit, and 20 μl of this RBC suspension was added to 1.0 ml NaCl solutions with osmolality ranging from 0 to 300 mOsm/Kg. The solutions were incubated at 37° C. for 30 min, centrifuged, and the absorbance of each supernatant measured at 540 nm. The absorbance for 0 mOsm/Kg solution was taken as 100% hemolysis. The osmolality of each solution was measured using a vapor pressure osmometer (Wescor, Logan, Utah).

TABLE 6
Hematological parameters of control (sham
loaded) and LMWP-ASNase loaded mice RBCs.

| Parameter | Control | Loaded |
|---|---|---|
| MCV (fL) | 58.3 ± 0.4 | 57.8 ± 0.4 |
| MCH (pg) | 17.3 ± 0.4 | 16.6 ± 0.3 |
| MCHC (g/dL) | 29.6 ± 0.4 | 28.7 ± 0.6 |

TABLE 7
Oxygen dissociation parameters of control
(sham loaded) and LMWP-ASNase loaded RBCs.

| | Oxygen Dissociation Parameters | | |
|---|---|---|---|
| | Control | Loaded | Reference |
| Hill Coefficient | 3.15 ± 0.32 | 3.07 ± 0.34 | 3.14 ± 0.12 |
| pO₅₀ (mmHg) | 37.44 ± 0.03 | 38.64 ± 0.02 | 40.0 ± 1.0 |

What is claimed is:

1. An erythrocyte population, comprising a quantifiable amount of erythrocytes, comprising:
i) a low molecular weight protamine peptide having at least 80% homology to the amino acid sequence VSRRRRRRGGRRRR (SEQ ID NO: 4); and
ii) L-asparaginase.

2. The erythrocyte population of claim 1, wherein the L-asparaginase is conjugated to the peptide.

3. The erythrocyte population of claim 2, wherein the conjugate comprises at least one said low molecular weight protamine peptide per L-asparaginase.

4. The erythrocyte population of claim,2, wherein the conjugate comprises 3-6 said peptide per L-asparaginase.

6. The erythrocyte population of claim 3, wherein the peptide comprises at least 14 amino acids, wherein the amino acid sequence of said peptide, comprises: VSRRRRRRGGRRRR (SEQ ID NO: 4).

7. The erythrocyte population of claim 4, wherein said population retains at least 50% of least one of the attributes and/or functionality, as compared to a normal, untreated erythrocyte population.

8. The erythrocyte population of claim 7, wherein said attributes and/or functionality, comprises:
i) structural integrity of the erythrocytes;
ii) biological and/or morphological integrity of the erythrocytes, comprising hematological values, comprising:
a) mean cell volume;
b) mean cell hemoglobin;
c) mean cell hemoglobin content;
iii) oxygen transport activity;
iv) oxygen dissociation;
v) osmotic fragility;
vi) energy (ATP)-involved metabolic activity; and/or
vii) scavenging of oxidative stress activity.

9. The erythrocyte population of claim 7, comprises a single erythrocyte.

10. A composition, comprising an L-asparaginase having the biological property to translocate across a biological membrane of an erythrocyte.

11. The composition of claim 10, comprising the L-asparaginase that is or was a conjugate to at least one low molecular weight protamine peptide having at least 80% homology to the amino acid sequence VSRRRRRRGGRRRR (SEQ ID NO: 4).

12. The composition of claim 10, wherein the erythrocyte, once comprising the L-asparaginase, substantially maintains at least one of the following properties:
i) structural integrity;
ii) biological integrity;
iii) morphological integrity; and
iv) functionality.

13. The composition of claim 12, wherein the erythrocyte substantially maintains at least its structural integrity and at least one of biological integrity, morphological integrity, and functionality.

14. A process of treating a patient suffering from cancer, comprising:
administering a therapeutic amount of erthrocytes comprising L-asparaginase to said patient.

15. The process of claim 14, wherein the L-asparaginase administered is or was conjugated to a low molecular weight protamine peptide having at least 80% homology to the amino acid sequence VSRRRRRRGGRRRR (SEQ ID NO: 4).

16. The process of claim 15, wherein the L-asparaginase is lysated from said conjugate.

17. The process of claim 16, wherein prior to the therapeutic administering of said erythrocytes, said process further comprises:
i) extracting a population of erythrocytes from said patient; and
ii) treating the extracted population of erythrocytes with L-asparaginase;

18. The process of claim 17, wherein the L-asparaginase used to treat the extracted erythrocytes is or was conjugated to a low molecular weight protamine peptide having at least 80% homology to SEQ ID NO: 4.

19. The process of claim 18, wherein the patient is suffering from leukemia.

20. The process of claim 14, comprising:
i) forming a conjugate, comprising:
a) one or more low molecular weight protamine peptides, wherein at least one of the peptides having at least 80% homology to the amino acid sequence VSRRRRRRGGRRRR (SEQ ID NO: 4); and
b) L-asparaginase;
ii) treating an erythrocyte population with said conjugate; and
iii) administering the conjugate treated erythrocyte population to said patient.
